(19) United States
(12) Patent Application Publication
(10) Pub. No.: US 2014/0235704 A1
(43) Pub. Date: Aug. 21, 2014

(54) THIOPHENE COMPOUNDS

(71) Applicant: Vertex Pharmaceuticals Incorporated, Boston, MA (US)

(72) Inventor: Brian Luisi, Mansfield, MA (US)

(73) Assignee: Vertex Pharmaceuticals Incorporated, Boston, MA (US)

(21) Appl. No.: 14/163,014

(22) Filed: Jan. 24, 2014

Related U.S. Application Data
(63) Continuation of application No. PCT/US2012/048261, filed on Jul. 26, 2012.
(60) Provisional application No. 61/511,647, filed on Jul. 26, 2011.
(60) Provisional application No. 61/511,644, filed on Jul. 26, 2011.
(60) Provisional application No. 61/545,751, filed on Oct. 11, 2011.
(60) Provisional application No. 61/512,079, filed on Jul. 27, 2011.
(60) Provisional application No. 61/511,648, filed on Jul. 26, 2011.
(60) Provisional application No. 61/623,144, filed on Apr. 12, 2012.
(60) Provisional application No. 61/511,643, filed on Jul. 26, 2011.

(51) Int. Cl. C07D 333/38; A61K 31/381

(52) U.S. Cl. CPC C07D 333/38; A61K 31/381
USPC 514/447; 435/184

(57) ABSTRACT

A co-crystal of Compound (1) includes Compound (1) and a co-crystal former selected from the group consisting of urea, nicotinamide, and isonicotinamide, wherein Compound (1) is characterized by the following structural formula:

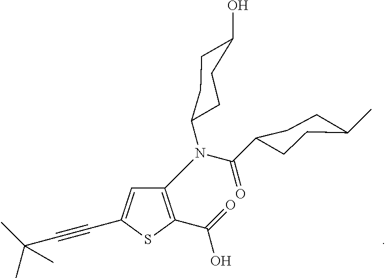 [SMILES: CC1CCC(C(=O)N(c2cc(C#CC(C)(C)C)sc2C(=O)O)C2CCC(O)CC2)CC1]

A pharmaceutical composition includes such a co-crystal of Compound (1) and at least one pharmaceutically acceptable carrier or excipient.

### RELATED APPLICATIONS

This application is a continuation of PCT Application No. PCT/US2012/048261, filed Jul. 26, 2012, which in turn claims priority to U.S. Provisional Application No. 61/511,644, filed Jul. 26, 2011; U.S. Provisional Application No. 61/511,647, filed Jul. 26, 2011; U.S. Provisional Application No. 61/512,079, filed Jul. 27, 2011; U.S. Provisional Application No. 61/511,643, filed Jul. 26, 2011; U.S. Provisional Application No. 61/511,648, filed Jul. 26, 2011; U.S. Provisional Application No. 61/545,751, filed Oct. 11, 2011; and U.S. Provisional Application No. 61/623,144, filed Apr. 12, 2012. The entire teachings of these applications are incorporated herein by reference.

### BACKGROUND OF THE INVENTION

Hepatitis C virus (HCV) is a positive-stranded RNA virus belonging to the Flaviviridae family and has closest relationship to the pestiviruses that include hog cholera virus and bovine viral diarrhea virus (BVDV). HCV is believed to replicate through the production of a complementary negative-strand RNA template. Due to the lack of efficient culture replication system for the virus, HCV particles were isolated from pooled human plasma and shown, by electron microscopy, to have a diameter of about 50-60 nm. The HCV genome is a single-stranded, positive-sense RNA of about 9,600 bp coding for a polyprotein of 3009-3030 amino-acids, which is cleaved co and post-translationally into mature viral proteins (core, E1, E2, p7, NS2, NS3, NS4A, NS4B, NS5A, NS5B). It is believed that the structural glycoproteins, E1 and E2, are embedded into a viral lipid envelope and form stable heterodimers. It is also believed that the structural core protein interacts with the viral RNA genome to form the nucleocapsid. The nonstructural proteins designated NS2 to NS5 include proteins with enzymatic functions involved in virus replication and protein processing including a polymerase, protease and helicase.

The main source of contamination with HCV is blood. The magnitude of the HCV infection as a health problem is illustrated by the prevalence among high-risk groups. For example, 60% to 90% of hemophiliacs and more than 80% of intravenous drug abusers in western countries are chronically infected with HCV. For intravenous drug abusers, the prevalence varies from about 28% to 70% depending on the population studied. The proportion of new HCV infections associated with post-transfusion has been markedly reduced lately due to advances in diagnostic tools used to screen blood donors.

Combination of pegylated interferon plus ribavirin is the treatment of choice for chronic HCV infection. This treatment does not provide sustained viral response (SVR) in a majority of patients infected with the most prevalent genotype (1a and 1b). Furthermore, significant side effects prevent compliance to the current regimen and may require dose reduction or discontinuation in some patients.

Antiviral agents against a HCV infection in general can be prepared in a variety of different forms. Such agents can be prepared so as to have a variety of different chemical forms including chemical derivatives or salts, or to have different physical forms. For example, they may be amorphous, may have different crystalline polymorphs, or may exist in different solvation or hydration states. By varying the forms, it may be possible to vary the physical properties thereof. For example, crystalline polymorphs may have different solubilities from one another. Pharmaceutical polymorphs can also differ in properties such as shelf-life, bioavailability, morphology, vapour pressure, density, colour, and compressibility. Such different forms may have different properties, in particular, as oral formulations. Specifically, it may be desirable to identify improved forms that exhibit improved properties, such as increased aqueous solubility and stability, better processability or preparation of pharmaceutical formulations, and increase of the bioavailability of orally-administered compositions. Such improved properties discussed above may be altered in a way which is beneficial for a specific therapeutic effect.

Variation of the crystalline state can be one of many ways in which to modulate the physical properties of antiviral agents to be more useful in treating HCV infection.

### SUMMARY OF THE INVENTION

The present invention generally relates to co-crystals of Compound (1), to methods of inhibiting or reducing the activity of HCV polymerase in a biological in vitro sample or in a subject, or of treating a HCV infection in a subject, which employ the co-crystals of Compound (1), and to a method of preparing the co-crystals of Compound (1):

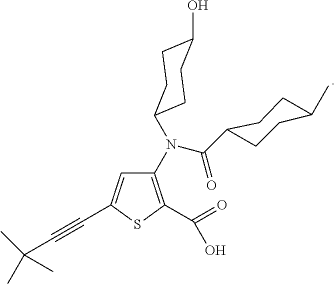 [SMILES: CC1CCC(C(=O)N(c2cc(C#CC(C)(C)C)sc2C(=O)O)C2CCC(O)CC2)CC1]

In one embodiment, the present invention is directed to a co-crystal comprising Compound (1) and a co-crystal former selected from the group consisting of urea, nicotinamide, and isonicotinamide.

In another embodiment, the present invention is directed to a pharmaceutical composition comprising a co-crystal of Compound (1) described herein and at least one pharmaceutically acceptable carrier or excipient.

In yet another embodiment, the present invention is directed to a method of inhibiting or reducing the activity of HCV polymerase in a biological in vitro sample. The method includes administering to the sample an effective amount of a co-crystal of Compound (1) described herein.

In yet another embodiment, the present invention is directed to a method of inhibiting or reducing the activity of HCV polymerase in a subject. The method includes administering to the subject an effective amount of a co-crystal of Compound (1) described herein.

In yet another embodiment, the present invention is directed to a method of treating a HCV infection in a subject. The method includes administering to the subject an effective amount of a co-crystal of Compound (1) described herein.

Methods of preparing co-crystals of Compound (1) described herein are also provided. The methods employ stirring a mixture of Compound (1) and a co-crystal former for a period of time to form the co-crystal.

Use of the co-crystals of Compound (1) described herein for inhibiting or reducing the activity of HCV polymerase in a biological in vitro sample or in a subject is also provided. Use of the co-crystals of Compound (1) described herein for treating a HCV infection in a subject is also provide.

The present invention also provides use of the co-crystals of Compound (1) described herein for the manufacture of a medicament for treating a HCV infection in a subject.

### SHORT DESCRIPTION OF DRAWINGS

FIGS. 1-3 show room temperature XRPD patterns of co-crystals of Compound (1) with urea, nicotinamide, and isonicotinamide, respectively.

### DETAILED DESCRIPTION OF THE INVENTION

Compound (1) represented by the following structural formula:

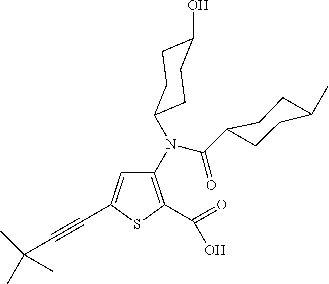 [SMILES: CC1CCC(C(=O)N(c2cc(C#CC(C)(C)C)sc2C(=O)O)C2CCC(O)CC2)CC1]

and pharmaceutically acceptable salts thereof are a NS5B polymerase inhibitors, and also described in WO 2008/058393.

The term “co-crystal” as used herein means a crystalline material comprised of two or more unique solids at room temperature, each containing distinctive physical characteristics, such as structure, melting point and heats of fusion, with the exception that, if specifically stated, the active pharmaceutical ingredient (API) may be a liquid at room temperature. The co-crystals typically comprise the API and a co-crystal former.

The co-crystal former may be H-bonded directly to the API or may be H-bonded to an additional molecule which is bound to the API. Other modes of molecular recognition may also be present including, pi-stacking, guest-host complexation and van der Waals interactions. The additional molecule may be H-bonded to the API or bound ionically or covalently to the API. The additional molecule could also be a different API. Solvates of API compounds that do not further comprise a co-crystal forming compound are not co-crystals according to the present invention. The co-crystals may however, include one or more solvate molecules in the crystalline lattice. That is, solvates of co-crystals, or a co-crystal further comprising a solvent or compound that is a liquid at room temperature, is included in the present invention, but crystalline material comprised of only one solid and one or more liquids (at room temperature) are not included in the present invention, with the previously noted exception of specifically stated liquid APIs.

In one aspect, the present invention is directed to a co-crystal comprising Compound (1) and a co-crystal former selected from the group consisting of urea, nicotinamide, and isonicotinamide. In one embodiment, the co-crystal former is urea. In another embodiment, the co-crystal former is nicotinamide. In yet another embodiment, the co-crystal former is isonicotinamide.

In one specific embodiment, the co-crystal comprising Compound (1) and urea as a co-crystal former (hereinafter “the urea co-crystal of Compound (1)”) is characterized as having an X-ray powder diffraction pattern with characteristic peaks expressed in 2-theta±0.2 at the following positions: 18.4, 12.1, 15.6, 20.1, 10.8, and 11.7. In another specific embodiment, the urea co-crystal of Compound (1) is characterized as having an X-ray powder diffraction pattern with characteristic peaks expressed in 2-theta±0.2 at the following positions with relative intensities in parentheses: 18.4 (100.0%), 12.1 (69.1%), 15.6 (65.0%), 20.1 (52.6%), 10.8 (46.5%), and 11.7 (44.1%). In yet another specific embodiment, the urea co-crystal of Compound (1) is characterized as having an endothermic peak in differential scanning calorimetry (DSC) at 190±2° C. In yet another specific embodiment, the urea co-crystal of Compound (1) is characterized as having X-ray powder diffraction pattern substantially the same as that shown in FIG. 1. The X-ray powder diffraction patterns are obtained at room temperature using Cu K alpha radiation.

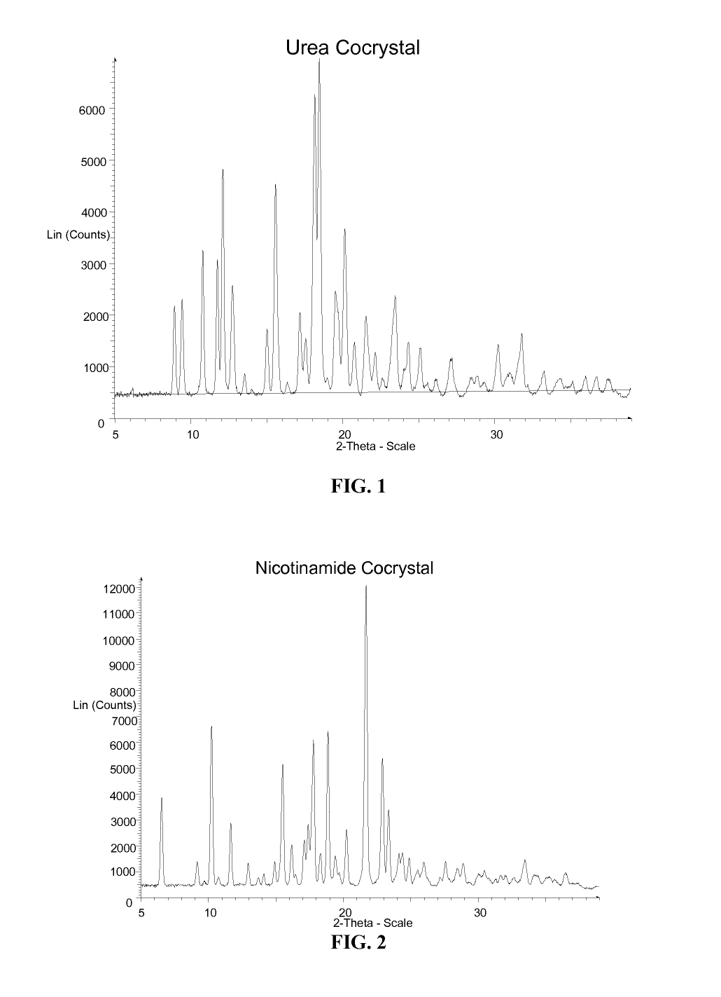

In yet another specific embodiment, the co-crystal comprising Compound (1) and nicotinamide as a co-crystal former (hereinafter “the nicotinamide co-crystal of Compound (1)”) is characterized as having an X-ray powder diffraction pattern with characteristic peaks expressed in 2-theta±0.2 at the following positions: 21.7, 10.2, 18.9, 17.8, 22.9, and 15.5. In another specific embodiment, the nicotinamide co-crystal of Compound (1) is characterized as having an X-ray powder diffraction pattern with characteristic peaks expressed in 2-theta±0.2 at the following positions with relative intensities in parentheses: 21.7 (100.0%), 10.2 (54.8%), 18.9 (53.2%), 17.8 (50.4%), 22.9 (44.6%), and 15.5 (42.5%). In yet another specific embodiment, the nicotinamide co-crystal of Compound (1) is characterized as having X-ray powder diffraction pattern substantially the same as that shown in FIG. 2. The X-ray powder diffraction patterns are obtained at room temperature using Cu K alpha radiation.

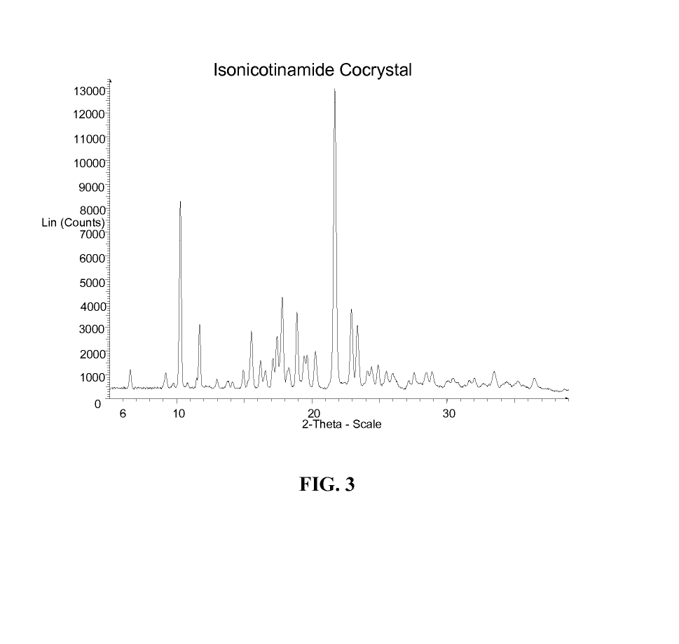

In yet another specific embodiment, the co-crystal comprising Compound (1) and isonicotinamide as a co-crystal former (hereinafter “the isonicotinamide co-crystal of Compound (1)”) is characterized as having an X-ray powder diffraction pattern with characteristic peaks expressed in 2-theta±0.2 at the following positions: 21.7, 10.2, 17.8, 22.9, 18.9, and 11.6. In yet another specific embodiment, the isonicotinamide co-crystal of Compound (1) is characterized as having an X-ray powder diffraction pattern with characteristic peaks expressed in 2-theta±0.2 at the following positions with relative intensities in parentheses: 21.7 (100.0%), 10.2 (63.6%), 17.8 (32.8%), 22.9 (28.9%), 18.9 (27.8%), and 11.6 (23.8%). In yet another specific embodiment, the isonicotinamide co-crystal of Compound (1) is characterized as having X-ray powder diffraction pattern substantially the same as that shown in FIG. 3. The X-ray powder diffraction patterns are obtained at room temperature using Cu K alpha radiation.

The present invention also provides methods of preparing the urea, nicotinamide, and isonicotinamide co-crystals of Compound (1). The co-crystals can be prepared by employing stirring a mixture of Compound (1) and the co-crystal former (urea, nicotinamide, or isonicotinamide) to form the co-crystal. In some specific embodiments, the mixture of Compound (1) and the co-crystal former is stitted in a suitable solvent at a suitable temperature (e.g., room temperature). In a specific embodiment, Compound (1) and the co-crystal former are in a 1:1 molar ratio. In another specific embodiment, examples of suitable solvents for preparing urea co-crystals of Compound (1) include dichloromethane and acetonitrile. In yet another specific embodiment, an example of suitable solvents for preparing nicotinamide and isonicotinamide co-crystals of Compound (1) includes acetonitrile.

The present invention encompasses co-crystals of Compound (1) described above in isolated, pure form, or in a mixture as a solid composition when admixed with other materials

Thus in one aspect there are provided co-crystals of Compound (1) in isolated solid form.

In a further aspect there are provided co-crystals of Compound (1) in pure form. The pure form means that a certain co-crystal of Compound (1) is over 95% (w/w), for example, over 98% (w/w), over 99% (w/w %), over 99.5% (w/w), or over 99.9% (w/w).

Assaying the solid phase for the presence of the co-crystals of Compound (1) and the co-crystal former may be carried out by conventional methods known in the art. For example, powder X-ray diffraction techniques can be used to assess the presence of co-crystals. Other techniques, used in an analogous fashion, include differential scanning calorimetry (DSC), thermogravimetric analysis (TGA), solid state NMR spectroscopy, and Raman spectroscopy. Single crystal X-ray diffraction may also be useful in identifying co-crystal structures.

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausolito: 1999, and “March's Advanced Organic Chemistry”, 5th Ed., Ed.: Smith, M. B. and March, J., John Wiley & Sons, New York: 2001, the entire contents of which are hereby incorporated by reference.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this invention, unless only one of the isomers is drawn specifically. As would be understood to one skilled in the art, a substituent can freely rotate around any rotatable bonds. For example, a substituent drawn as

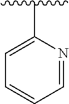 [SMILES: *c1ccccn1]

also represents

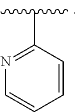 [SMILES: *c1ccccn1]

Therefore, single stereochemical isomers as well as enantiomeric, diastereomeric, cis/trans, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays. Such compounds, especially deuterium (D) analogs, can also be therapeutically useful.

The compounds described herein are defined herein by their chemical structures and/or chemical names. Where a compound is referred to by both a chemical structure and a chemical name, and the chemical structure and chemical name conflict, the chemical structure is determinative of the compound's identity.

It will be appreciated by those skilled in the art that the compounds in accordance with the present invention can exists as stereoisomers (for example, optical (+ and −), geometrical (cis and trans) and conformational isomers (axial and equatorial). All such stereoisomers are included in the scope of the present invention.

It will be appreciated by those skilled in the art that the compounds in accordance with the present invention can contain a chiral center. The compounds of formula may thus exist in the form of two different optical isomers (i.e. (+) or (−) enantiomers). All such enantiomers and mixtures thereof including racemic mixtures are included within the scope of the invention. The single optical isomer or enantiomer can be obtained by method well known in the art, such as chiral HPLC, enzymatic resolution and chiral auxiliary.

In one embodiment, the compounds in accordance with the present invention are provided in the form of a single enantiomer at least 95%, at least 97% and at least 99% free of the corresponding enantiomer.

In a further embodiment, the compounds in accordance with the present invention are in the form of the (+) enantiomer at least 95% free of the corresponding (−) enantiomer.

In a further embodiment, the compounds in accordance with the present invention are in the form of the (+) enantiomer at least 97% free of the corresponding (−) enantiomer.

In a further embodiment, the compounds in accordance with the present invention are in the form of the (+) enantiomer at least 99% free of the corresponding (−) enantiomer.

In a further embodiment, the compounds in accordance with the present invention are in the form of the (−) enantiomer at least 95% free of the corresponding (+) enantiomer.

In a further embodiment, the compounds in accordance with the present invention are in the form of the (−) enantiomer at least 97% free of the corresponding (+) enantiomer.

In a further embodiment the compounds in accordance with the present invention are in the form of the (−) enantiomer at least 99% free of the corresponding (+) enantiomer.

The co-crystals of Compound (1) can be used for treating or preventing a Flaviviridae viral infection in a host by administering to the host a therapeutically effective amount of at least one co-crystal of Compound (1) according to the invention described herein.

The terms “subject,” “host,” or “patient” includes an animal and a human (e.g., male or female, for example, a child, an adolescent, or an adult). Preferably, the “subject,” “host,” or “patient” is a human.

In one embodiment, the viral infection is chosen from Flavivirus infections. In one embodiment, the Flavivirus infection is Hepatitis C virus (HCV), bovine viral diarrhea virus (BVDV), hog cholera virus, dengue fever virus, Japanese encephalitis virus or yellow fever virus.

In one embodiment, the Flaviviridea viral infection is hepatitis C viral infection (HCV), such as HCV genotype 1, 2, 3, or 4 infection.

In one embodiment, the co-crystals of Compound (1) can be used for treatment of HCV genotype 1 infection. The HCV can be genotype 1a or genotype 1b.

In one embodiment, the co-crystals of Compound (1) can be used for treating or preventing a Flaviviridae viral infection in a host comprising administering to the host a therapeutically effective amount of at least one co-crystal of Compound (1) according to the invention described herein, and further comprising administering at least one additional agent chosen from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES).

In one embodiment, there is provided a method for inhibiting or reducing the activity of viral polymerase in a host comprising administering a therapeutically effective amount of a co-crystal of Compound (1) according to the invention described herein.

In one embodiment, there is provided a method for inhibiting or reducing the activity of viral polymerase in a host comprising administering a therapeutically effective amount of a co-crystal of Compound (1) according to the invention described herein and further comprising administering one or more viral polymerase inhibitors.

In one embodiment, viral polymerase is a Flaviviridae viral polymerase.

In one embodiment, viral polymerase is a RNA-dependant RNA-polymerase.

In one embodiment, viral polymerase is HCV polymerase.

In one embodiment, viral polymerase is HCV NS5B polymerase.

In one embodiment, the present invention provides a pharmaceutical composition comprising a co-crystal of Compound (1) according to the invention described herein and at least one pharmaceutically acceptable carrier, adjuvant, or vehicle, which includes any solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undue, undesired reactions or side-effects upon the administration to a subject. Standard pharmaceutical formulation techniques can be employed.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The co-crystals of Compound (1) described above, and pharmaceutically acceptable compositions thereof can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Specifically, the compositions are administered orally, intraperitoneally or intravenously.

Any orally acceptable dosage form including, but not limited to, capsules, tablets, aqueous suspensions or solutions, can be used for the oral administration. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Liquid dosage forms for oral administration include, but are not limited to, pharmaceutically acceptable emulsions, microemulsions, solutions, suspensions, syrups and elixirs. In addition to the active compounds (the co-crystals of Compound (1) described above), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The co-crystals of Compound (1) described above can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Sterile injectable forms may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In order to prolong the effect of the co-crystals of Compound (1) administered, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the active compound in biodegradable polymers such as polylactide-polyglycolide. Depending upon the ratio of the active compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

When desired the above described formulations adapted to give sustained release of the active ingredient may be employed.

Compositions for rectal or vaginal administration are specifically suppositories which can be prepared by mixing the active compound with suitable non-irritating excipients or carriers such as cocoa butter, polyethylene glycol or a suppository wax which are solid at ambient temperature but liquid at body temperature and therefore melt in the rectum or vaginal cavity and release the active compound.

Dosage forms for topical or transdermal administration include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, transdermal patches, which have the added advantage of providing controlled delivery of a compound to the body, can also be used. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Alternatively, the co-crystals of Compound (1) and pharmaceutically acceptable compositions thereof may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The co-crystals of Compound (1) and pharmaceutically acceptable compositions thereof can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose. The amount of the active compound in a unit dosage form will vary depending upon, for example, the host treated, and the particular mode of administration, for example, from 0.01 mg/kg body weight/day to 100 mg/kg body weight/day.

It will be appreciated that the amount of a co-crystal of Compound (1) according to the invention described herein required for use in treatment will vary not only with the particular compound selected but also with the route of administration, the nature of the condition for which treatment is required and the age and condition of the patient and will be ultimately at the discretion of the attendant physician or veterinarian. In general however a suitable dose will be in the range of from about 0.1 to about 750 mg/kg of body weight per day, for example, in the range of 0.5 to 60 mg/kg/day, or, for example, in the range of 1 to 20 mg/kg/day.

The desired dose may conveniently be presented in a single dose or as divided dose administered at appropriate intervals, for example as two, three, four or more doses per day.

The co-crystals of Compound (1) can be formulated as a pharmaceutical composition which further includes one or more additional agents chosen from viral serine protease inhibitors, viral NS5A inhibitors, iral polymerase inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES). For example, the pharmaceutical composition may include the active compound(s); one or more additional agents select from non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon and ribavirin; and at least one pharmaceutically acceptable carrier or excipient.

The co-crystals of Compound (1) can be employed as a combination therapy in combination with one or more additional agents chosen from viral serine protease inhibitors, viral polymerase inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES).

The co-crystals of Compound (1) and additional agent can be administered sequentially. Alternatively, the active compounds and additional agent can be administered simultaneously. The combinations referred to above may conveniently be presented for use in the form of a pharmaceutical formulation and thus pharmaceutical formulations comprising a combination as defined above together with a pharmaceutically acceptable carrier therefore comprise a further aspect of the invention.

The term “viral serine protease inhibitor” as used herein means an agent that is effective to inhibit the function of the viral serine protease including HCV serine protease in a mammal. Inhibitors of HCV serine protease include, for example, those compounds described in WO 99/07733 (Boehringer Ingelheim), WO 99/07734 (Boehringer Ingelheim), WO 00/09558 (Boehringer Ingelheim), WO 00/09543 (Boehringer Ingelheim), WO 00/59929 (Boehringer Ingelheim), WO 02/060926 (BMS), WO 2006039488 (Vertex), WO 2005077969 (Vertex), WO 2005035525 (Vertex), WO 2005028502 (Vertex) WO 2005007681 (Vertex), WO 2004092162 (Vertex), WO 2004092161 (Vertex), WO 2003035060 (Vertex), of WO 03/087092 (Vertex), WO 02/18369 (Vertex), or WO98/17679 (Vertex).

The term “viral polymerase inhibitors” as used herein means an agent that is effective to inhibit the function of a viral polymerase including an HCV polymerase in a mammal. Inhibitors of HCV polymerase include non-nucleosides, for example, those compounds described in: WO 03/010140 (Boehringer Ingelheim), WO 03/026587 (Bristol Myers Squibb); WO 02/100846 A1, WO 02/100851 A2, WO 01/85172 AI (GSK), WO 02/098424 A1 (GSK), WO 00/06529 (Merck), WO 02/06246 A1 (Merck), WO 01/47883 (Japan Tobacco), WO 03/000254 (Japan Tobacco) and EP 1 256 628 A2 (Agouron).

Furthermore other inhibitors of HCV polymerase also include nucleoside analogs, for example, those compounds described in: WO 01/90121 A2 (Idenix), WO 02/069903 A2 (Biocryst Pharmaceuticals Inc.), and WO 02/057287 A2 (Merck/Isis) and WO 02/057425 A2 (Merck/Isis).

Specific examples of nucleoside inhibitors of an HCV polymerase, include R1626, R1479 (Roche), R7128 (Roche), MK-0608 (Merck), R1656, (Roche-Pharmasset) and Valopicitabine (Idenix). Specific examples of inhibitors of an HCV polymerase, include JTK-002/003 and JTK-109 (Japan Tobacco), HCV-796 (Viropharma), GS-9190(Gilead), and PF-868,554 (Pfizer).

The term “viral NS5A inhibitor” as used herein means an agent that is effective to inhibit the function of the viral NS5A protease in a mammal. Inhibitors of HCV NS5A include, for example, those compounds described in WO2010/117635, WO2010/117977, WO2010/117704, WO2010/1200621, WO2010/096302, WO2010/017401, WO2009/102633, WO2009/102568, WO2009/102325, WO2009/102318, WO2009020828, WO2009020825, WO2008144380, WO2008/021936, WO2008/021928, WO2008/021927, WO2006/133326, WO2004/014852, WO2004/014313, WO2010/096777, WO2010/065681, WO2010/065668, WO2010/065674, WO2010/062821, WO2010/099527, WO2010/096462, WO2010/091413, WO2010/094077, WO2010/111483, WO2010/120935, WO2010/126967, WO2010/132538, and WO2010/122162. Specific examples of HCV NS5A inhibitors include: EDP-239 (being developed by Enanta); ACH-2928 (being developed by Achillion); PPI-1301 (being developed by Presido Pharmaceuticals); PPI-461 (being developed by Presido Pharmaceuticals); AZD-7295 (being developed by AstraZeneca); GS-5885 (being developed by Gilead); BMS-824393 (being developed by Bristol-Myers Squibb); BMS-790052 (being developed by Bristol-Myers Squibb)

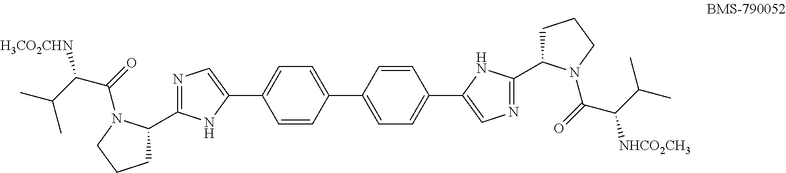

(Gao M. et al. Nature, 465, 96-100 (2010): nucleoside or nucleotide polymerase inhibitors, such as PSI-661 (being developed by Pharmasset), PSI-938 (being developed by Pharmasset), PSI-7977 (being developed by Pharmasset), INX-189 (being developed by Inhibitex), JTK-853 (being developed by Japan Tobacco), TMC-647055 (Tibotec Pharmaceuticals), RO-5303253 (being developed by Hoffmann-La Roche), and IDX-184 (being developed by Idenix Pharmaceuticals).

The term “viral helicase inhibitors” as used herein means an agent that is effective to inhibit the function of a viral helicase including a Flaviviridae helicase in a mammal.

“Immunomodulatory agent” as used herein means those agents that are effective to enhance or potentiate the immune system response in a mammal. Immunomodulatory agents include, for example, class I interferons (such as alpha-, beta-, delta- and omega-interferons, x-interferons, consensus interferons and asialo-interferons), class II interferons (such as gamma-interferons) and pegylated interferons.

Exemplary immunomudulating agents, include, but are not limited to: thalidomide, IL-2, hematopoietins, IMPDH inhibitors, for example Merimepodib (Vertex Pharmaceuticals Inc.), interferon, including natural interferon (such as OMNIFERON, Viragen and SUMIFERON, Sumitomo, a blend of natural interferon's), natural interferon alpha (ALFERON, Hemispherx Biopharma, Inc.), interferon alpha n1 from lymphblastoid cells (WELLFERON, Glaxo Wellcome), oral alpha interferon, Peg-interferon, Peg-interferon alfa 2a (PEGASYS, Roche), recombinant interferon alpha 2a (ROFERON, Roche), inhaled interferon alpha 2b (AERX, Aradigm), Peg-interferon alpha 2b (ALBUFERON, Human Genome Sciences/Novartis, PEGINTRON, Schering), recombinant interferon alfa 2b (INTRON A, Schering), pegylated interferon alfa 2b (PEG-INTRON, Schering, VIRAFERONPEG, Schering), interferon beta-1a (REBIF, Serono, Inc. and Pfizer), consensus interferon alpha (INFERGEN, Valeant Pharmaceutical), interferon gamma-1b (ACTIMMUNE, Intermune, Inc.), un-pegylated interferon alpha, alpha interferon, and its analogs, and synthetic thymosin alpha 1 (ZADAXIN, SciClone Pharmaceuticals Inc.).

The term “class I interferon” as used herein means an interferon selected from a group of interferons that all bind to receptor type 1. This includes both naturally and synthetically produced class I interferons. Examples of class I interferons include alpha-, beta-, delta- and omega-interferons, tau-interferons, consensus interferons and asialo-interferons. The term “class II interferon” as used herein means an interferon selected from a group of interferons that all bind to receptor type II. Examples of class II interferons include gamma-interferons.

Antisense agents include, for example, ISIS-14803.

Specific examples of inhibitors of HCV NS3 protease, include BILN-2061 (Boehringer Ingelheim) SCH-6 and SCH-503034/Boceprevir (Schering-Plough), VX-950/telaprevir (Vertex) and ITMN-B (InterMune), GS9132 (Gilead), TMC-435350(Tibotec/Medivir), ITMN-191 (InterMune), MK-7009 (Merck).

Inhibitor internal ribosome entry site (IRES) includes ISIS-14803 (ISIS Pharmaceuticals) and those compounds described in WO 2006019831 (PTC therapeutics).

In one embodiment, the additional agents for the compositions and combinations include, for example, ribavirin, amantadine, merimepodib, Levovirin, Viramidine, and maxamine.

In one embodiment, the additional agent is interferon alpha, ribavirin, silybum marianum, interleukine-12, amantadine, ribozyme, thymosin, N-acetyl cysteine or cyclosporin.

In one embodiment, the additional agent is interferon alpha 1A, interferon alpha 1 B, interferon alpha 2A, or interferon alpha 2B. Interferon is available in pegylated and non pegylated forms. Pegylated interferons include PEGASYS™ and Peg-intron™

The recommended dose of PEGASYS™ monotherapy for chronic hepatitis C is 180 mg (1.0 mL vial or 0.5 mL prefilled syringe) once weekly for 48 weeks by subcutaneous administration in the abdomen or thigh.

The recommended dose of PEGASYS™ when used in combination with ribavirin for chronic hepatitis C is 180 mg (1.0 mL vial or 0.5 mL prefilled syringe) once weekly.

Ribavirin is typically administered orally, and tablet forms of ribavirin are currently commercially available. General standard, daily dose of ribavirin tablets (e.g., about 200 mg tablets) is about 800 mg to about 1200 mg. For example, ribavirn tablets are administered at about 1000 mg for subjects weighing less than 75 kg, or at about 1200 mg for subjects weighing more than or equal to 75 kg. Nevertheless, nothing herein limits the methods or combinations of this invention to any specific dosage forms or regime. Typically, ribavirin can be dosed according to the dosage regimens described in its commercial product labels.

The recommended dose of PEG-lntron™ regimen is 1.0 mg/kg/week subcutaneously for one year. The dose should be administered on the same day of the week.

When administered in combination with ribavirin, the recommended dose of PEG-lntron is 1.5 micrograms/kg/week.

The combinations referred to above may conveniently be presented for use in the form of a pharmaceutical formulation and thus pharmaceutical formulations comprising a combination as defined above together with a pharmaceutically acceptable carrier therefore comprise a further aspect of the invention. The individual components for use in the method of the present invention or combinations of the present invention may be administered either sequentially or simultaneously in separate or combined pharmaceutical formulations.

In one embodiment, the additional agent is interferon α 1A, interferon α 1B, interferon α 2A, or interferon α 2B, and optionally ribavirin.

When co-crystals of Compound (1) is used in combination with at least one second therapeutic agent active against the same virus, the dose of each compound may be either the same as or differ from that when the compound is used alone. Appropriate doses will be readily appreciated by those skilled in the art.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXEMPLIFICATION

### Example 1

### General Methods of XRPD and DSC Measurements

DSC Measurements

DSC was conducted on a TA Instruments model Q2000 V24.3 calorimeter (Asset Tag V014080). Approximately 1-2 mg of solid sample was placed in an aluminum hermetic DSC pan with a crimped lid with a pinhole. The sample cell was heated under nitrogen purge at 10° C. per minute to 300° C.

Bruker D8 Discover XRPD Experimental Details.

The XRPD patterns were acquired at room temperature in reflection mode using a Bruker D8 Discover diffractometer (Asset Tag V012842) equipped with a sealed tube source and a Hi-Star area detector (Bruker AXS, Madison, Wis.). The X-Ray generator was operating at a voltage of 40 kV and a current of 35 mA. The powder sample was placed in an aluminum holder. Two frames were registered with an exposure time of 120 s each. The data were subsequently integrated over the range of 4°-40° 2θ with a step size of 0.02° and merged into one continuous pattern.

### Example 2

### Formation of Compound (1)

### Method A

Compound (1) can be prepared as described in WO 2008/058393:

### Preparation of 5-(3,3-Dimethyl-but-1-ynyl)-3-[(trans-4-hydroxy-cyclohexyl)-(trans-4-methyl-cyclohexanecarbonyl)-amino]-thiophene-2-carboxylic acid

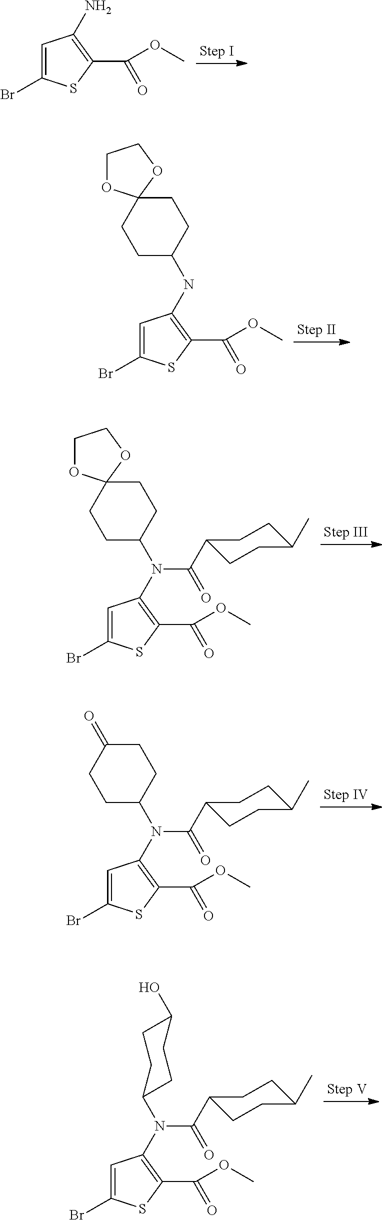 [SMILES: COC(=O)c1sc(Br)cc1N ; COC(=O)c1sc(Br)cc1NC1CCC2(CC1)OCCO2 ; COC(=O)c1sc(Br)cc1N(C(=O)C1CCC(C)CC1)C1CCC2(CC1)OCCO2 ; COC(=O)c1sc(Br)cc1N(C(=O)C1CCC(C)CC1)C1CCC(=O)CC1 ; COC(=O)c1sc(Br)cc1N(C(=O)C1CCC(C)CC1)C1CCC(O)CC1]

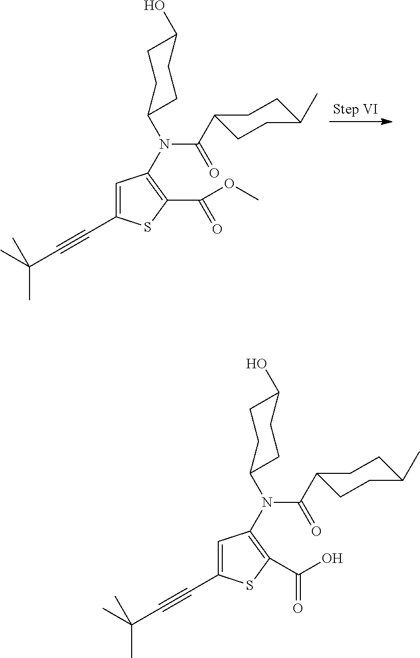

### Step I

A suspension of 3-amino-5-bromo-thiophene-2-carboxylic acid methyl ester (17.0 g, 72.0 mmol) in dry THF (21 mL) is treated with 1,4-cyclohexanedione monoethylene ketal (11.3 mg, 72.0 mmol), followed by dibutyltin dichloride (1.098 g, 3.6 mmol). After 5 min, phenyl silane (9.74 mL, 79.2 mmol) is added and the reaction mixture is stirred over-night at room temperature. After concentration, the residue is dissolved in EtOAc washed with NaHCO₃ then brine. The organic layer is separated, dried on Na₂SO₄, filtered and concentrated. The crude material is diluted in hexane (500 mL). After filtration, the mother liquor is evaporated to dryness to give 5-bromo-3-(1,4-dioxa-spiro[4.5]dec-8-ylamino)-thiophene-2-carboxylic acid methyl ester (24.79 g, 92% yield). Ref: WO2004/052885

### Step II

A—Preparation of trans-4-methylcyclohexyl carboxylic acid chloride:

Oxalyl chloride (2M in DCM, 117 mL) is added drop wise to a suspension of trans-4-methylcyclohexyl carboxylic acid (16.6 g, 117 mmol) in DCM (33 mL) and DMF (0.1 mL), and the reaction mixture is stirred 3 h at room temperature. DCM is removed under reduced pressure and the residue is co-evaporated with DCM. The residue is dissolved in toluene to make a 1M solution.

### B—Preparation of the Target Compound:

The 1M solution of trans-4-methylcyclohexyl carboxylic acid chloride is added to a solution of 5-bromo-3-(1,4-dioxa-spiro[4.5]dec-8-ylamino)-thiophene-2-carboxylic acid methyl ester (24.79 g, 65 mmol) in toluene (25 mL) followed by pyridine (5.78 mL, 71.5 mmol). The resulting mixture is then stirred for 16 h at reflux. The reaction mixture is diluted with toluene (60 mL) and cooled down to 5° C. After the addition of pyridine (12 mL) and MeOH (5.6 mL), the mixture is stirred 2 h at 5° C. The white suspension is filtered off and the toluene is added to the mother liquor. The organic phase is washed with 10% citric acid, aq. Sat NaHCO₃, dried (Na₂SO₄) and concentrated. The residue is triturated in boiling hexane (1500 mL). The reaction mixture is allowed to cool down to room temperature. The reaction flask is immersed into ice bath, and stirred for 30 min; white solid is filtered off, and washed with cold hexane (225 mL). The solid is purified by silica gel column chromatography using 20% EtOAc:hexane as eluent to furnish the final compound 5-bromo-3-[(1,4-dioxa-spiro[4.5]dec-8-yl)-(trans-4-methyl-cyclohexanecarbonyl)-amino]-thiophene-2-carboxylic acid methyl ester (10.5 g, 32%).

### Step III

5-Bromo-3-[(1,4-dioxa-spiro[4.5]dec-8-yl)-(trans-4-methylcyclohexane-carbonyl)-amino]-thiophene-2-carboxylic acid methyl ester (8.6 g, 17 mmol) is dissolved in tetrahydrofuran (100 mL) and treated with 3N HCl solution (50 mL). The reaction is stirred at 40° C. for 3 h. The reaction mixture is evaporated under reduced pressure. The residue is dissolved in EtOAc and washed with aq. sat. NaHCO₃ solution. The organic layer is separated, dried on Na₂SO₄, filtered and concentrated to give 5-bromo-3-[(trans-4-methyl-cyclohexanecarbonyl)-(4-oxo-cyclohexyl)-amino]-thiophene-2-carboxylic acid methyl ester as a solid (7.4 g, 95%).

### Step IV

To a cold (0° C.) solution of 5-bromo-3-[(trans-4-methyl-cyclohexanecarbonyl)-(4-oxo-cyclohexyl)-amino]-thiophene-2-carboxylic acid methyl ester (5.9 g, 12.9 mmol) in 50 mL of MeOH under a N₂ atmosphere, NaBH₄ (250 mg, 6.4 mmol) is added portion wise (approx. 30 min). After the addition is completed and checked for reaction completion by TLC (hexane:EtOAc 1:1), 10 mL of HCl 2% is added and stirred for 15 min. The reaction mixture is concentrated under vacuum to dryness. The reaction mixture is recuperated with water (25 mL) and extracted with EtOAC. The organic phases are combined and dried over MgSO₄ and concentrated to dryness. The residue is purified by silica gel column chromatography using EtOAc:hexane (1:1) as eluent to obtain 5-bromo-3-[(trans-4-hydroxy-cyclohexyl)-(trans-4-methyl-cyclohexane-carbonyl)-amino]-thiophene-2-carboxylic acid methyl ester (4.5 g, 77% yield) as a solid.

### Step V

To a solution of compounds 5-bromo-3-[(trans-4-hydroxy-cyclohexyl)-(trans-4-methyl-cyclohexanecarbonyl)-amino]-thiophene-2-carboxylic acid methyl ester (500 mg, 1.09 mmol) and 3,3-Dimethyl-but-1-yne (385 mg, 4.69 mmol) in DMF (0.5 mL), triethylamine (1.06 mL) and tris(dibenzylideneacetone) dipalladium (0) (70 mg, 0.08 mmol) are added and the reaction mixture is stirred under reflux conditions for 16 h under a N₂ atmosphere. DMF and triethylamine are removed under reduced pressure and the residue is partitioned between water and ethyl acetate. The organic layer is separated, dried (Na₂SO₄), concentrated and the residue is purified by column chromatography using ethyl acetate and hexane (1:2) as eluent to obtain 5-(3,3-dimethyl-but-1-ynyl)-3-[(trans-4-hydroxy-cyclohexyl)-(trans-4-methyl-cyclohexanecarbonyl)-amino]-thiophene-2-carboxylic acid methyl ester as a solid, 330 mg (66%).

### Step VI

5-(3,3-Dimethyl-but-1-ynyl)-3-[(trans-4-hydroxy-cyclohexyl)-(trans-4-methyl-cyclohexanecarbonyl)-amino]-thiophene-2-carboxylic acid methyl ester (0.10 g, 0.22 mmol) is dissolved in a 3:2:1 mixture of THF:methanol:H₂O (5.0 mL) and treated with a 1N solution of LiOH.H₂O (0.65 mL, 0.65 mmol). After 2 h of stirring at 60° C., the reaction mixture is concentrated under reduced pressure on a rotary evaporator. The mixture is partitioned between ethyl acetate and water. The water layer is acidified using 0.1 N HCl. The EtOAc layer is separated and dried over Na₂SO₄. Filtration and removal of the solvent under reduced pressure on a rotary evaporator followed by purification by column chromatography using methanol and dichloromethane (1:9) as eluent to obtain 5-(3,3-dimethyl-but-1-ynyl)-3-[(trans-4-hydroxy-cyclohexyl)-(trans-4-methyl-cyclohexanecarbonyl)-amino]-thiophene-2-carboxylic acid as a solid, 30 mg (30%). ESI (M−H): 444.3. ¹H NMR (400 MHz, DMSO-d₆) δ 0.58 (m, 1H), 0.74 (q, J=6.53 Hz, 1H), 0.81 (ddd, J=12.86, 12.49, 3.19 Hz, 1H), 1.18 (m, 5H), 1.28 (s, 3H), 1.42 (m, 1H), 1.55 (m, 3H), 1.61 (m, 1H), 1.73 (m, 2H), 1.81 (m, 2H), 3.19 (m, 1H), 4.26 (m, 1H), 4.49 (bs, 1H), 7.14 (s, 1H), 13.45 (bs, 1H).

### Method B

### Preparation of 5-(3,3-Dimethyl-but-1-ynyl)-3-[(trans-4-hydroxy-cyclohexyl)-(trans-4-methyl-cyclohexanecarbonyl)-amino]-thiophene-2-carboxylic acid

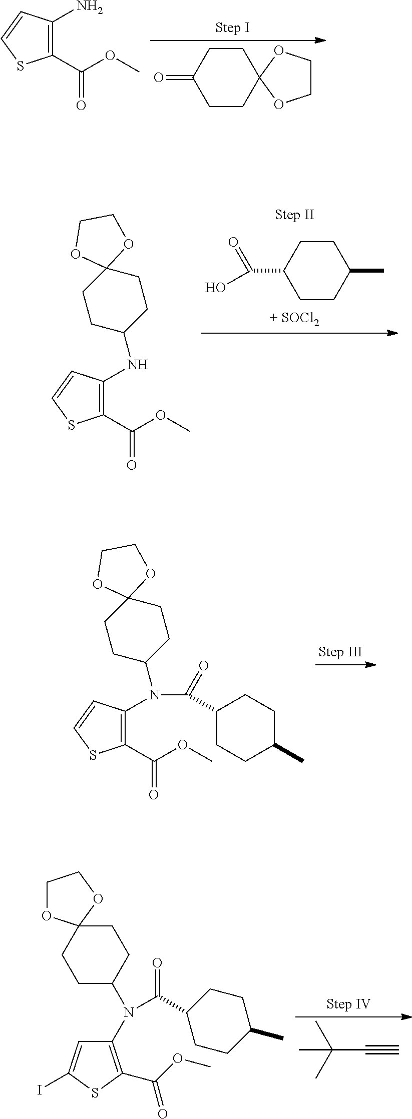

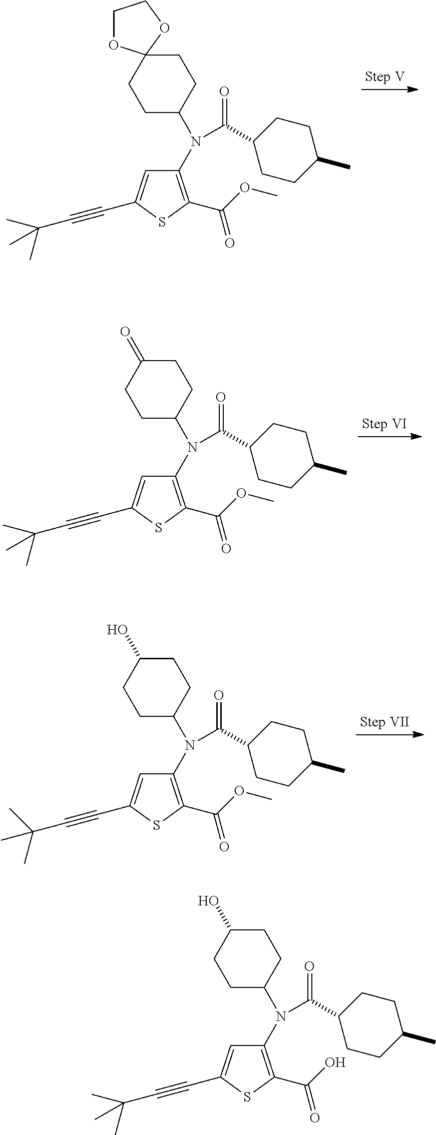

### Step I

A suspension of 3-amino-thiophene-2-carboxylic acid methyl ester (5.0 g, 31.85 mmol) in dry THF (9 mL) is treated with 1,4-cyclohexanedione monoethylene ketal (5.0 g, 32.05 mmol), followed by dibutyltin dichloride (482 mg, 1.59 mmol). After 5 min, phenyl silane (4.3 mL, 34.96 mmol) is added and the reaction mixture is stirred overnight at room temperature. After concentration, the residue is dissolved in EtOAc and washed with NaHCO₃ followed by brine. The organic layer is separated, dried (Na₂SO₄), filtered and concentrated. The residue is purified by column chromatography using 30% ethyl acetate in hexane as eluent to give 3-(1,4-dioxa-spiro[4.5]dec-8-ylamino)-thiophene-2-carboxylic acid methyl ester (4.5 g, 47% yield).

### Alternative Procedure:

3-Amino-thiophene-2-carboxylic acid methyl ester (1 eq.) is dissolved in dichloromethane followed by 1,4-cyclohexanedione monoethylene acetal (2 eq.) to obtain a slightly yellow solution. This solution is added to the suspension of NaBH(OAc)₃ (2.2 eq.) in dichloromethane. Acetic acid (2.4 eq.) is added dropwise over a period of 15 min. The resulting suspension is stirred at 20-25° C. under N₂ for 24 h. The reaction is quenched by adding water and stirred for 1 h. Dichloromethane layer is separated, treated with water again and stirred for another 1 h. The dichloromethane layer is separated and added to a saturated NaHCO₃ solution, stirred at 20-25° C. for 20 min. Some of the white residual solids are filtered and then the organic layer is separated, dried (Na₂SO₄) and evaporated. Methanol is added to the residue and evaporated to dryness. The residue is taken in of methanol and stirred for 2 h at 0° C. The suspension is vacuum-filtered and the resulting filtered cake is washed with cold methanol. The white solid is dried under vacuum at 35-40° C. for approximately 20 h to afford the title compound.

### Step II

A. Preparation of trans-4-methylcyclohexyl carboxylic acid chloride

Oxalyl chloride (2M in dichloromethane, 17 mL) is added dropwise to a suspension of trans-4-methylcyclohexyl carboxylic acid (2.3 g, 16.2 mmol) in dichloromethane (5 mL) and DMF (0.1 mL). The reaction mixture is stirred for 3 h at room temperature. The volatiles are removed under reduced pressure to obtain the crude acid chloride which is used directly for the next reaction.

B. trans-4-Methylcyclohexyl carboxylic acid chloride is added to a solution of 3-(1,4-dioxa-spiro[4.5]dec-8-ylamino)-thiophene-2-carboxylic acid methyl ester (2.4 g, 8.08 mmol) in toluene (18 mL) followed by pyridine (0.7 mL). The resulting mixture is then stirred for 16 h at reflux. The reaction mixture is diluted with toluene (7 mL) and cooled to 5° C. After the addition of pyridine (1.5 mL) and MeOH (0.8 mL), the mixture is stirred 2 h at 5° C. The white solid is filtered and washed with toluene. The filtrate is washed with 10% citric acid, aq. NaHCO₃, dried (Na₂SO₄) and concentrated. The solid is purified by silica gel column chromatography using 20% EtOAc:hexane as eluent to obtain 3-[(1,4-dioxa-spiro[4.5]dec-8-yl)-(trans-4-methyl-cyclohexanecarbonyl)-am-ino]-thiophene-2-carboxylic acid methyl ester (2.3 g, 68%).

### Alternative Procedure:

To a solution of trans-4-methylcyclohexyl carboxylic acid (1.8 eq.) in toluene under nitrogen is added anhydrous DMF. The reaction mixture is stirred and thionyl chloride (2.16 eq.) is added over 3-5 min. The mixture is then stirred for 3 h at rt. When the reaction is completed, toluene is added to the reaction mixture. The solution is then evaporated under reduced nitrogen pressure to half of its volume. The solution is dissolved in toluene to obtain a 1N acid chloride solution.

3-(1,4-Dioxa-spiro[4.5]dec-8-ylamino)-thiophene-2-carboxylic acid methyl ester (1 eq.) and pyridine (2 eq.) are added to the acid chloride (1N) solution. The reaction mixture is stirred at reflux for 15 h. Once the reaction is completed, the reaction mixture is cooled to room temperature, and then methanol and toluene are added to it. The reaction mixture is stirred for 1 h at rt and a saturated aqueous solution of NaHCO₃ is added. The organic layer is separated, dried (Na₂SO₄) and evaporated to about 4 volumes of solvent. To the solution are added 4 volumes of heptane while stirring. The reaction flask is immersed into an ice bath and stirred for 120 min; a beige solid is filtered off and washed with cold heptane, then dried over night in the vacuum oven to obtain the title compound.

### Step III

n-BuLi (2 eq.) is added dropwise for 10 min to a cold (−40° C.) solution of diisopropylamine (1 eq.) in dry THF. The reaction mixture is stirred at the same temperature for 30 min. Then a solution of 3-[(1,4-dioxa-spiro[4.5]dec-8-yl)-(trans-4-methyl-cyclohexane-carbonyl)-a-mino]-thiophene-2-carboxylic acid methyl ester (1 eq.) in THF is added dropwise (35 min) keeping the internal temperature around −40.degree. C. The reaction mixture is stirred for 30 min and a solution of iodine (2 eq.) in THF is added dropwise, stirred for 30 min at the same temperature before being added a sat. solution of NH₄Cl. The reaction mixture is diluted with ethyl acetate and water. The organic layer is separated and washed with 5% sodium thiosulfate solution. The organic layer is separated, dried (Na₂SO₄) and evaporated to a suspension and then added heptane. The suspension is stirred at 0.degree. C. for 30 min, filtered and washed with heptane to obtain 3-[(1,4-dioxa-spiro[4.5]dec-8-yl)-(trans-4-methyl-cyclo-hexanecarbonyl)-a-mino]-5-iodo-thiophene-2-carboxylic acid methyl ester. MS found (electrospray): (M+H): 548.21

### Step IV

To a 25 mL RBF under nitrogen, 3-[(1,4-dioxa-spiro[4.5]dec-8-yl)-(trans-4-methyl-cyclohexanecarbonyl)-am-ino]-5-iodo-thiophene-2-carboxylic acid methyl ester (1 eq.), copper iodide (0.025 eq.) and tris(dibenzylideneacetone) dipalladium (0) (0.01 eq.) are taken. DMF, triethylamine (2.5 eq.) and 3,3-dimethyl-but-1-yne (2 eq.) are added and the reaction mixture is stirred at 40° C. for 2 h under a N₂ atmosphere. The reaction mixture is filtered on celite and washed with ethyl acetate. The solution is diluted with water and extracted 2 times with ethyl acetate. The organic phases are combined and washed 3 times with water. The organic layer is separated, dried (Na₂SO₄), evaporated to about 2 mL and then 8 mL of heptane is added. It is evaporated to 2-4 mL and cooled in an ice bath. The formed white solid is filtered, washed with heptane and dried in oven to obtain 5-(3,3-dimethyl-but-1-ynyl)-3-[(1,4-dioxa-spiro[4.5]dec-8-yl)-(trans-4-me-thyl-cyclohexanecarbonyl)-amino]-thiophene-2-carboxylic acid methyl ester.

### Step V

5-(3,3-Dimethyl-but-1-ynyl)-3-[(1,4-dioxa-spiro[4.5]dec-8-yl)-(trans-4-methyl-cyclohexanecarbonyl)-amino]-thiophene-2-carboxylic acid methyl ester (1 eq.) is dissolved in tetrahydrofuran and treated with 3.6 N HCl solution. The reaction is stirred at 40° C. for 5 h. Water is then added and the reaction mixture is cooled to room temperature. The reaction mixture is extracted with ethyl acetate (2.times.50 mL). The combined extracts are washed with 25 mL of aqueous saturated NaHCO₃ and 2×50 mL of water. The organic layer is concentrated to a thick oil and 50 mL of heptane is added to the mixture to precipitate the desired compound which is filtered to give of 5-(3,3-dimethyl-but-1-ynyl)-3-[(trans-4-methyl-cyclohexanecarbonyl)-(4-ox-o-cyclohexyl)-amino]-thiophene-2-carboxylic acid methyl ester.

### Step VI

5-(3,3-Dimethyl-but-1-ynyl)-3-[(trans-4-methyl-cyclohexanecarbonyl)-(4-oxo-cyclohexyl)-amino]-thiophene-2-carboxylic acid methyl ester (1 eq.) is dissolved in THF. Water is added to the reaction mixture and cooled to −25.degree. C. NaBH.sub.4 (0.5 eq.) is added portion wise maintaining the temperature below −20.degree. C. The mixture is stirred for 2 h at −25.degree. C., 2N HCl is then added and the solution is warmed to room temperature. The phases are separated and the aqueous layer is washed with EtOAC. The organic phases are combined and washed with brine and dried over Na.sub.2SO.sub.4 and concentrated to dryness to give 5-(3,3-dimethyl-but-1-ynyl)-3-[(4-hydroxy-cyclohexyl)-(trans-4-methyl-cyc-lohexanecarbonyl)-amino]-thiophene-2-carboxylic acid methyl ester as a 93:7 mixture of isomers. The crude cis/trans mixture is recrystallized in methanol to obtain >99% the trans isomer.

### Step VII

The same procedure as reported earlier (Method A, step VI) is followed to obtain 5-(3,3-dimethyl-but-1-ynyl)-3-[(trans-4-hydroxy-cyclohexyl)-(trans-4-meth-yl-cyclohexanecarbonyl)-amino]-thiophene-2-carboxylic acid. MS found (electrospray): (M−H): 444.3 ¹H NMR (400 MHz, DMSO-d₆) δ 0.58 (m, 1H), 0.74 (q, J=6.53 Hz, 1H), 0.81 (ddd, J=12.86, 12.49, 3.19 Hz, 1H), 1.18 (m, 5H), 1.28 (s, 3H), 1.42 (m, 1H), 1.55 (m, 3H), 1.61 (m, 1H), 1.73 (m, 2H), 1.81 (m, 2H), 3.19 (m, 1H), 4.26 (m, 1H), 4.49 (bs, 1H), 7.14 (s, 1H), 13.45 (bs, 1H).

### Method C

### 1. Step 1

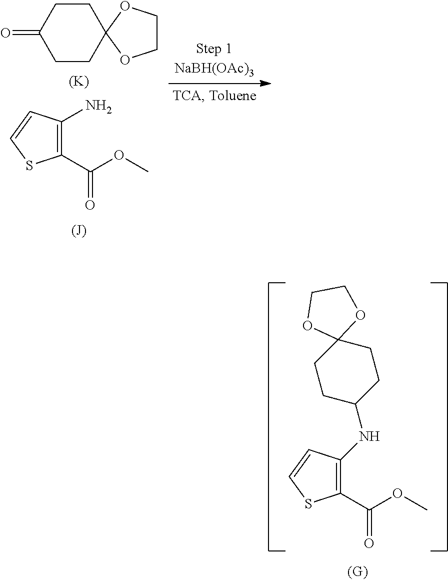

Compounds J (50.0 g, 1.0 eq.), K (52.2 g, 1.05 eq), and NaBH(OAc)₃ (118.0 g, 1.75 eq) were added to a reactor followed by toluene (600 mL, 12 vol). Started agitation then adjusted the internal temperature to 0-5° C. The mixture was a heterogeneous suspension of white solids. Then was added trichloroacetic acid (TCA, 52.0 g, 1.0 eq) in toluene (150 mL, 3 vol) to the stirring mixture over 1 h while controlling the internal temperature to between 0-5° C. The reaction mixture was warmed to 20-25° C., and then stirred for 2-4 hours at 20-25° C. under an atmosphere of nitrogen. The reaction progress was monitored by HPLC.

Upon completion of reaction, the reaction mixture was transferred into a solution of K₂CO₃ (307.7 g, 7.0 eq) in DI water (375 mL, 7.5 vol). The biphasic mixture was stirred and then the phases were separated. The organic phase was washed with aqueous solution of K₂CO₃ (175.9 g, 4.0 eq) in DI water (375 mL, 7.5 vol), then with aqueous solution of NaCl (20.4 g, 1.1 eq) in DI water (375 mL, 7.5 vol). The organic phase was separated. The batch volume was reduced by distillation (to 250 mL (5 vol) on a rotary evaporator at a bath temperature of ≦40° C.) and the resulting crude solution of Compound G in toluene was used in the next step (HPLC: 98.29% AUC chemical purity). Compound G: ¹H NMR (400 MHz, DMSO-d₆) δ 1.45 (m, 2H), 1.64 (m, 4H), 1.88 (m, 2H), 3.56 (m, 1H), 3.72 (s, 3H), 3.87 (m, 4H), 6.70 (d, J=6.8 Hz, 1H), 6.90 (d, J=4.4 Hz, 1H), 7.70 (d, J=4.4 Hz, 1H).

### 2. Step 2

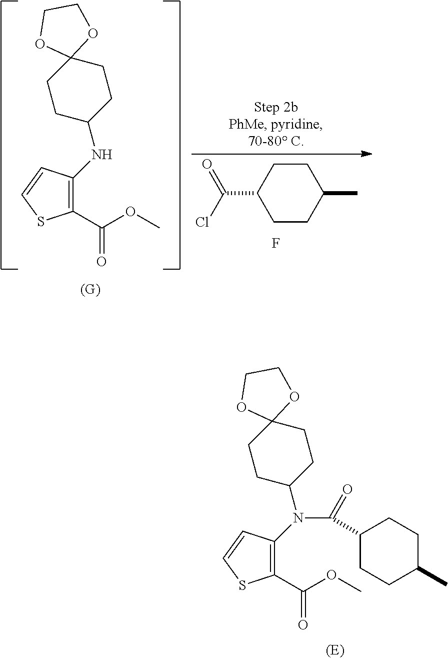

2.1. Step 2b: Using trans-methylcyclohexane carbonyl chloride (Compound F)

To the solution of compound G in toluene (94.6 g, 250 mL, 5.0 vol) from previous step was added toluene (410 mL, 8.2 vol) and pyridine (64.0 mL, 2.5 eq). Agitation was started and the internal temperature was adjusted to 20-25° C. Compound F (102.2 g, 2.0 eq) was added over 0.5 h. The batch was heated to 95-100° C. once the addition had complete. The reaction progress was monitored by HPLC. Upon completion of reaction, the batch was cooled to 30-35° C., then methanol (189 mL, 3.8 vol) was added over 45 minutes and the batch was stirred for 1-2 hours. Added DI water (189 L, 3.8 vol) to the batch at 30-35° C. then it was allowed to stir at 60-70° C. for 1-2 hours. The mixture was heated to 55-60° C. then stirred for 1 h.

The phases were separated. DI water (189 mL, 3.8 vol) was added at 55-60° C. then stirred for 1 hour. The toluene phase was concentrated by distillation. The batch was heated to 78-83° C. (e.g, 80° C.), then n-heptane (473 mL, 9.5 vol) was added to toluene solution over 1-3 hours, and the batch was then stirred at 90-95° C. over 2 hours. The batch was cooled to 20-25° C. over 5 hours, followed by stirring at 20-25° C. for 1-12 hours. The solids were filtered. The filter cake was washed with n-heptane (190 mL, 3.8 vol) and dried under vacuum at 40-45° C. for 10-20 hours. The isolated compound E was analyzed by HPLC, GC, and Karl Fischer titration. Overall yield for Steps 1 & 2=113.5 g, 84.1%. HPLC: 99.39% AUC chemical purity (Typical purity >98.0%). Compound E: ¹H NMR (400 MHz, DMSO-d₆) δ0.48 (m, 1H), 0.63 (m, 1H), 0.74 (d, J=6.4 Hz, 3H), 0.98 (m, 1H), 1.22 (m, 2H), 1.36 (m, 1H), 1.52-1.67 (m, 10H), 1.77 (m, 2H), 3.75-3.78 (m, 4H), 3.76 (s, 3H), 4.44 (m, 1H), 7.11 (d, J=5.2 Hz, 1H), 8.00 (d, J=5.2 Hz, 1H).

2.2. Step 2a: Using trans-methylcyclohexane carboxylic acid (Compound H)

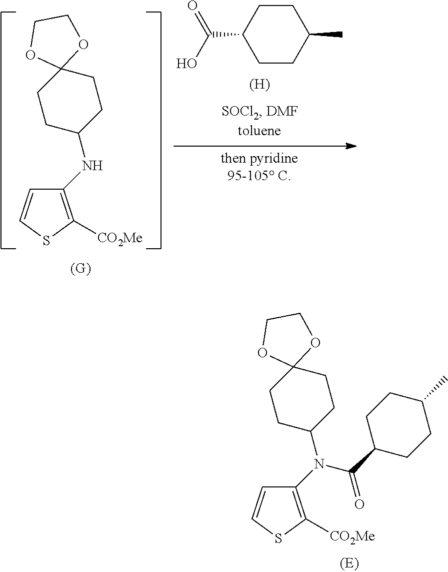

Compound H (633 g, 2.0 eq) was charged to a reactor-1 under a N₂ atmosphere. Toluene (1.33 L, 3.8 vol) was then added to the reactor, followed by DMF (1.73 mL, 0.01 eq), then agitation was started. SOCl₂ (325 mL, 2.0 eq) was added slowly over 30 minutes. The internal temperature was adjusted to 33-37° C. (e.g., 35° C.). The solution was stirred at 33-37° C. for 2 hours. The mixture was cooled to 20-25° C., transferred to a rotary evaporator, and then concentrated to 3.8 vol (˜1.3 L). Toluene (665 mL, 1.9 vol) was then added to the concentrate and the resulting batch was concentrated to 3.8 vol (˜1.3 L).

Compound G in toluene (662 g, 1.75 L, 5.0 vol) was charged to a reactor-2 under N₂ atmosphere. Toluene (4.97 L, 14.2 vol) and pyridine (448 mL, 2.5 eq) was added to the reactor-2. Agitation was started and the internal temperature was adjusted to 20-25° C.

The solution of reactor-1 (acid chloride obtained above) in toluene was added to the reactor-2 over 1 hour. The reaction mixture was heated to 95-105° C. once the addition had complete. An IPC sample was taken after 24-30 h and analyze for Compound G consumption by HPLC.

The reaction mixture was then cooled to 25-30° C. MeOH (665 mL, 1.9 vol) was added to the reaction mixture over 45 minutes. DI water (1.33 L, 3.8 vol) was then added to the reaction mixture at 25-30° C. The mixture was heated to 55-60° C. then stirred for 1 hour. Stopped agitation and allowed the phases to separate for 10 minutes. The upper organic layer was separated and the aqueous layer was set aside. DI water (1.33 L, 3.8 vol) was added to the reaction mixture at 55-60° C. then stirred for 1 hour. Stopped agitation and allowed the phases to separate for 10 minutes. The upper organic layer was separated and the aqueous layer was set aside. The solution was transferred (while it remained at ˜60° C.) to a rotary evaporator and concentrated to 5.7 vol (˜2 L). Heptane (3.3 L, 5.0 vol) was then added to the suspension at ˜60° C. The suspension was cooled to 20-25° C. while stirring over 5 hours. The suspension was filtered. The cake was washed twice with heptane (665 mL, 1.9 vol). The solids were dried on the filter under vacuum. Overall yield for Steps 1 & 2=805.2 g, 85.8% as a white solid. HPLC: 99.15% AUC chemical purity. Compound E: ¹H NMR (400 MHz, DMSO-d₆) δ0.48 (m, 1H), 0.63 (m, 1H), 0.74 (d, J=6.4 Hz, 3H), 0.98 (m, 1H), 1.22 (m, 2H), 1.36 (m, 1H), 1.52-1.67 (m, 10H), 1.77 (m, 2H), 3.75-3.78 (m, 4H), 3.76 (s, 3H), 4.44 (m, 1H), 7.11 (d, J=5.2 Hz, 1H), 8.00 (d, J=5.2 Hz, 1H).

### 3. Step 3

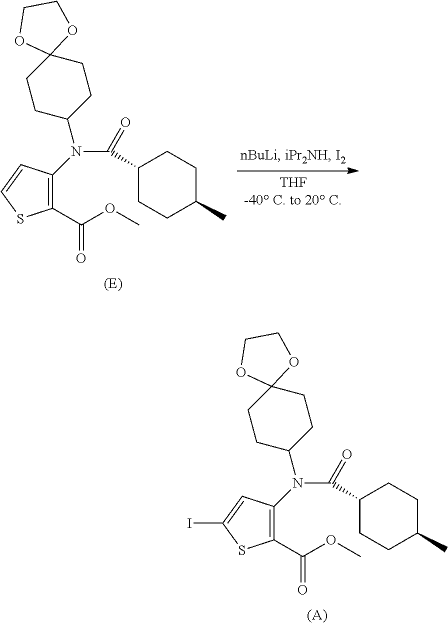

Anhydrous THF (1.0 L, 2.0 vol) and anhydrous diisopropylamine (258 mL, 1.55 eq) were added to Reactor-1. The solution was cooled to −50° C. to −40° C. Once the desired temperature was achieved, a 1.6M solution of n-butyl lithium in hexanes (1.11 L, 1.50 eq) was added at a rate such that the internal temperature remained below −40° C. After the addition had completed, the solution stirred at −50° to −40° C. for another 2 hours.

Compound E (500 g, 1.0 eq) and anhydrous THF (5.0 L, 10.0 vol) were charged to Reactor-2. The resulting solution was added to Reactor-1 over 1 hour at a rate such that the internal temperature remained below −40° C. A solution of iodine (361 g, 1.20 eq) in THF (500 mL, 1.0 vol) was added to the cold reaction mixture at a rate such that the internal temperature remained below −40° C. The reaction mixture was at −50° to −40° C. for 1 hour. The reaction progress was monitored by HPLC.

Upon completion of reaction, the batch was warmed to 0-5° C. and transferred to a solution of NaHSO₃ (617 g, 5.0 eq) in DI water (2.5 L, 5.0 vol) cooled to 0-5° C. Dichloromethane (1.5 L, 3.0 vol) was added to the suspension. The biphasic mixture was stirred for 1 hour while warming to 20-25° C. The phases were separated. The aqueous phase was washed with dichloromethane. The organic phases were combined and washed twice with aqueous solution of NH₄CL (634 g, 10.0 eq) in DI water (1.9 L, 5.0 vol), followed by wash with water. The batch volume was reduced by distillation. Solvent switch to toluene was performed: added toluene (1.5 L, 3.0 vol) again then concentrated to 3.0 vol (˜1.5 L). Toluene (5.0 L, 10.0 vol) was then added to the resulting concentrate and the mixture was heated to 95-100° C. until a homogenous solution was obtained. Added heptane (5.0 L, 10.0 vol) at 95-100° C. to the toluene solution, then the mixture was cooled to 20-25° C. over 6 hours. The suspension was filtered. The cake was washed twice with heptane (500 mL, 1.0 vol). The solids were dried on the filter under vacuum. The isolated compound A was analyzed by HPLC, GC, and Karl Fischer titration. Yield for Steps 3=520.5 g, 80.2% as a beige solid. HPLC: Typical >97.0% AUC chemical purity. Compound A: ¹H NMR (400 MHz, DMSO-d₆) δ 0.54 (m, 1H), 0.65 (m, 1H), 0.76 (d, J=6.8 Hz, 3H), 1.00 (m, 1H), 1.22 (m, 2H), 1.30 (m, 1H), 1.44-1.68 (m, 10H), 1.60-1.69 (m, 4H), 1.77 (m, 2H), 3.74 (s, 3H), 3.77 (m, 4H), 4.40 (m, 1H), 7.46 (s, 1H).

### 4. Step 4

A. Method A1

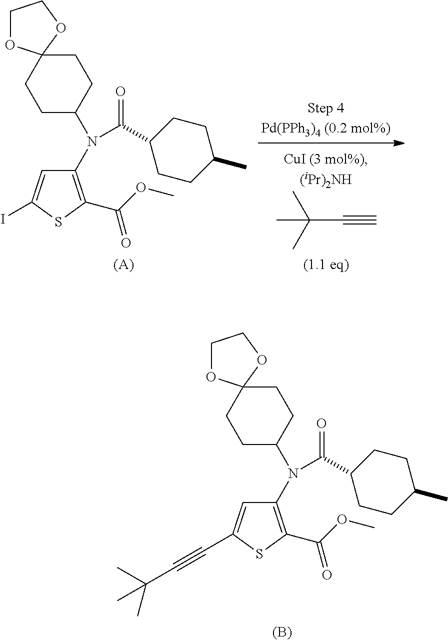

A jacketed 1 L 3-neck reactor was fitted with a nitrogen inlet then charged with Compound (A) (112.7 g, 205.9 mmol). CuI (1.18 g, 6.18 mmol) and Pd(PPh₃)₄ (457.9 mg, 0.412 mmol) were added to the reactor. The reactor was purged with a stream of nitrogen then anhydrous 2-methyltetrahydrofuran (789 mL) was added. The mixture was stirred for 15 mins at 20-25° C. Anhydrous diisopropylamine (52.09 g, 72.15 mL, 514.8 mmol) and tert-butylacetylene (18.59 g, 27.0 mL, 226.5 mmol) were added to the reactor. This mixture was then stirred between 20-25° C. Complete conversion after stirring for 4 h had been reached according to HPLC. The mixture was cooled to 10° C. The organic phase was then washed with 12.6 wt % aqueous oxalic acid for at least 3 hours then the phases were split. Activated carbon (22.5 g) was added to the reaction mixture. The suspension was stirred at 20-25° C. for not less than 12 hours. The mixture was filtered over celite. The filter cake was washed with 2-butanone (563.5 mL) and the filtrate was added to the organic phase. Analysis of the organic solution by HPLC showed Compound (B) purity to be 99.56% AUC. This solution is typically used directly in the next step. Compound (B): ¹H NMR (400 MHz, DMSO-d₆) δ 0.52-0.59 (m, 1H), 0.61-0.70 (m, 1H), 0.76 (d, J=6.4 Hz, 3H), 0.88-1.03 (m, 1H), 1.15-1.37 (m, 4H), 1.31 (s, 9H)S, 1.41-1.68 (m, 9H), 1.74-1.85 (m, 2H), 3.75-3.81 (m, 4H), 3.75 (s, 3H), 4.39-4.42 (m, 1H), 7.27 (s, 1H).

B. Method A2

A jacketed 1 L 3-neck reactor was fitted with a nitrogen inlet then charged with Compound (A) (63.94 g). CuI (667.3 mg, 0.03 eq) and Pd(PPh₃)₄ (269.9 mg, 0.002 eq) were added to the reactor. The reactor was purged with a stream of nitrogen then methyl t-butyl ether (MtBE) (7 vol) was added. The mixture was stirred for 15 mins at 20-25° C. Anhydrous diisopropylamine (40.9 mL, 2.5 eq) was added to the stirring mixture while maintaining the internal temperature between 20-25° C. and stirred the batch for NLT 15 minutes. tert-Butylacetylene (16.7 mL, 1.2 eq) were added to the reactor. This mixture was then stirred between 20-25° C. Complete conversion after stirring for 4 h had been reached according to HPLC. The mixture was cooled to 10° C. The organic phase was then washed with 12.6 wt % aqueous oxalic acid dehydrate (383.6 mL, 6 vol) was added while maintaining the batch temperature below 20-25° C. The batch temperature was then adjusted to 20-25° C. and the biphasic mixture was stirred for at least 3 hours at this temperature. The phases were then allowed to separate for at least 30 minutes. The organic phase was then again washed with aqueous oxalic acid dehydrate (6 wt % 383.6 mL, 6 vol) while maintaining the batch temperature below 20-25° C. The biphasic mixture was stirred for at least 1 hour at this temperature. Then the phases were split. Activated carbon (6.4 g-12.8 g, 10-20 wt % with respect to Compound A) was added to the reaction mixture. The suspension was stirred at 20-25° C. for not less than 12 hours. The mixture was filtered over celite. The filter cake was washed with MtBE (192 mL, 3 vol) and the filtrate was added to the organic phase. This solution is typically used directly in the next step.

C. Method B

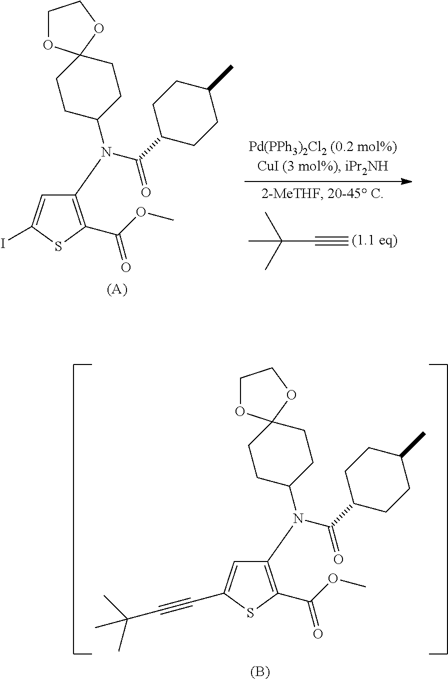

A jacketed 3 L 3-neck reactor was fitted with a nitrogen inlet then charged with Compound (A) (20.00 g, 36.53 mmol). CuI (208.7 mg, 1.096 mmol) and Pd(PPh₃)₂Cl₂ (51.28 mg, 0.07306 mmol) were added to the reactor. The reactor was purged with a stream of nitrogen then anhydrous 2-methyltetrahydrofuran (140.0 mL) was added. The mixture was stirred for 15 mins at 20-25° C. Anhydrous diisopropylamine (9.241 g, 12.80 mL, 91.32 mmol) and tert-butylacetylene (3.751 g, 5.452 mL, 45.66 mmol) were added to the reactor. This mixture was then stirred between 20-25° C. (20.9° C.) (a suspension is formed). The mixture was then heated to 45° C. for 6 h. An HPLC analysis showed conversion to be 99.77%. Heptane (140.0 mL) was added while cooling to 20° C. over 4 h. The suspension was filtered. The filtrate was washed with an aqueous oxalic acid dihydrate solution (120 mL of 15% w/v, 142.8 mmol). The phases were split then the organic phase was washed with aqueous NH₄Cl (120 mL of 10% w/v, 224.3 mmol), aqueous NaHCO₃ (120 mL of 7% w/w), and water (120.0 mL). Residual metals were scavenged by addition of 2.0 g charcoal (10% wt of VRT-0921870) followed by stirring at 20-25° C. for 5 h. The suspension was then filtered over celite. The celite bed was washed with 2-methyltetrahydrofuran (40.0 mL). Analysis of the organic solution by HPLC showed Compound (B) purity to be 99.47% AUC.

D. Method C

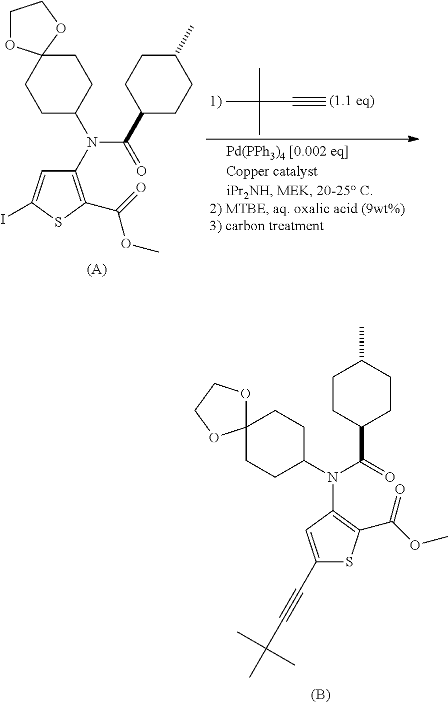

To a round bottom flask equipped with mechanical stirring, N₂ bubbler and thermocouple, was added Compound (A) [1.0 eq], copper catalyst, Pd (PPh₃)_{4 [}0.002 eq] and MEK [7 volume]. The reaction solution was stirred at room temperature to dissolve followed by addition of iPr₂NH [2.5 equiv] and tert-butylacetylene [1.1 equiv]. The reaction solution was stirred at 20-25° C. The reaction conversion (conv [%]) was monitored via LC. For the copper catalyst, CuI (99.9%), CuI (98%), CuCl, and CuBr were tested:
CuI (for both 99.9% and 98%): with 0.03 equiv of CuI, over 95% conversion into Compound (B) after about 2 hours' reaction time; with 0.025 equiv of CuI, over 90% conversion into Compound (B) after about 5 hours' reaction time; with 0.02 equiv of CuI, over 90% conversion into Compound (B) after about 5 hours' reaction time; with 0.015 equiv of CuI, over 90% conversion into Compound (B) after about 5 hours' reaction time; with 0.01 equiv of CuI, over 75% conversion into Compound (B) after about 5 hours' reaction time;
CuCl: with 0.03 equiv of CuCl, over 99% conversion into Compound (B) after about 2 hours' reaction time; with 0.025 equiv of CuI, approximately 100% conversion into Compound (B) after about 2 hours' reaction time; with 0.02 equiv of CuCl, over 90% conversion into Compound (B) after about 2 hours' reaction time; with 0.015 equiv of CuCl, over 95% conversion into Compound (B) after about 2 hours' reaction time; with 0.01 equiv of CuCl, approximately 100% conversion into Compound (B) after about 20 hours' reaction time;
CuBr: with 0.03 equiv of CuBr, over 99% conversion into Compound (B) after about 22 hours' reaction time; with 0.025 equiv of CuBr, over 85% conversion into Compound (B) after about 22 hours' reaction time; with 0.02 equiv of CuBr, over 95% conversion into Compound (B) after about 22 hours' reaction time; with 0.015 equiv of CuBr, over 70% conversion into Compound (B) after about 22 hours' reaction time; with 0.01 equiv of CuBr, over 80% conversion into Compound (B) after about 22 hours' reaction time.

### 5. Step 5

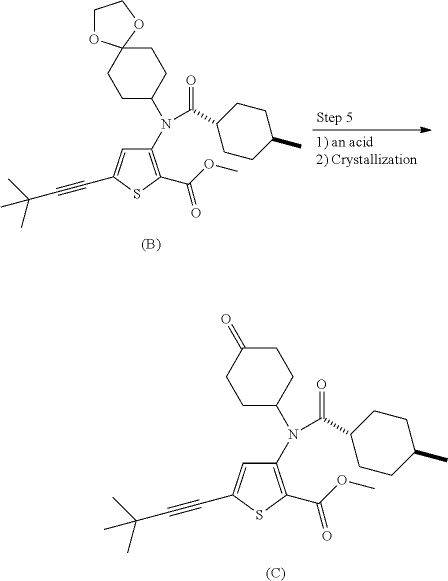

### A. Method A

A jacketed 1 L 4-neck reactor was fitted with a nitrogen inlet then charged with a solution of Compound (B) (22.9 g, 45.65 mmol) in 2-butanone (˜250 mL), then heated to 60° C. The reactor was purged with a stream of nitrogen then an aqueous solution of 2N HCl (175 mL) was added. The mixture was stirred at 60° C. for 4 hours. The stirring was stopped and the lower aqueous phase was removed. Agitation was started again followed by the addition of fresh aqueous solution of 2N HCl (175 mL). The mixture continued to stir at 60° C. until the conversion (99% by HPLC) had reached equilibrium (approximately another 2.5 hours). After cooling to 20° C., the lower aqueous phase was removed. The organic phase was then washed with 10 wt % aqueous NH₄Cl then the phases were split. The organic phase was then distilled to ˜115 mL. Acetone (115 mL) was added then the batch was concentrated to ˜115 mL. This procedure of acetone addition followed by distillation was repeated twice more. Water (57.3 mL) was added to the organic phase at 20° C. then the mixture stirred for 2 hours. Water was added to the organic phase at 20° C. over 2 hours then the mixture stirred for an additional hour. The solids were filtered and washed with 1:1 MeOH/H₂O (25 mL), then dried in a vacuum oven with nitrogen bleed at 60° C. for 24 hours to give 19.8 g (95% yield) of Compound (C). ¹H NMR (400 MHz, DMSO-d₆) δ 0.56-0.68 (m, 2H), 0.76 (d, J=6.4 Hz, 3H), 1.19-1.30 (m, 4H), 1.30 (s, 9H), 1.46-1.60 (m, 6H), 1.83-1.89 (m, 2H), 2.05-2.18 (m, 3H), 2.47-2.55 (m, 1H), 3.76 s, 3H), 4.77-4.85 (m, 1H), 7.30 (s, 1H).

### B. Method B

A jacketed 1 L 4-neck reactor was fitted with a nitrogen inlet then charged with a solution of Compound (B) (103.3 g, 1.0 eq based on 100% yield in Step 4) in 2-butanone (˜1.03 L, approximately 10 vol total batch volume), then heated to 57° C.-62° C. (e.g., 60° C.). The reactor was purged with a stream of nitrogen then an aqueous solution of 2N HCl (723 mL, 7 vol based on 103.3 g of Compound (B)) was added over about 10 minutes while maintaining the batch temperature at 57° C.-62° C. (e.g., 60° C.). The mixture was stirred at 57° C.-62° C. (e.g., 60° C.) for 5 hours. The stirring was stopped and the lower aqueous phase was removed. Agitation was started again followed by the addition of fresh aqueous solution of 2N HCl (310 mL, 3 vol based on 103.3 g of Compound (B)). The mixture continued to stir at 57° C.-62° C. (e.g., 60° C.) until the conversion (99% by HPLC) had reached equilibrium (approximately another 2.5 hours). After cooling to 20-25° C., the agitation was stopped and phases were allowed to separate for at least 30 minutes. An aqueous NH₄Cl (10 wt %, 517 mL, 5 vol) was then added while maintaining the batch temperature at 20-25° C. The biphasic mixture was stirred for at least 30 minutes at 20-25° C. Then the phases were split. The organic phase was then distilled to 471 mL by vacuum distillation with a maximum jacketed temperature of 60° C. Acetone (471.1 mL) was added then the batch was concentrated to ˜471 mL. This procedure of acetone addition followed by distillation was repeated twice more. Water (235.6 mL, 2.28 vol) was added to the organic phase at 20° C. then the mixture stirred for 2 hours. Additional water (235.6 mL, 2.28 vol) was added to the organic phase at 20° C. over 2 hours then the mixture stirred for an additional hour. The solids were filtered and washed with a 1:1 mixture of acetone/H₂O (vol:vol, 103 mL: 103 mL), then dried in a vacuum oven with nitrogen bleed at 60° C. for 24 hours to give 19.8 g (99.5% yield) with overall purity of 98.0%) of Compound (C).

### 6. Step 6

### A. Method A: Using LiAlH(OtBu)₃

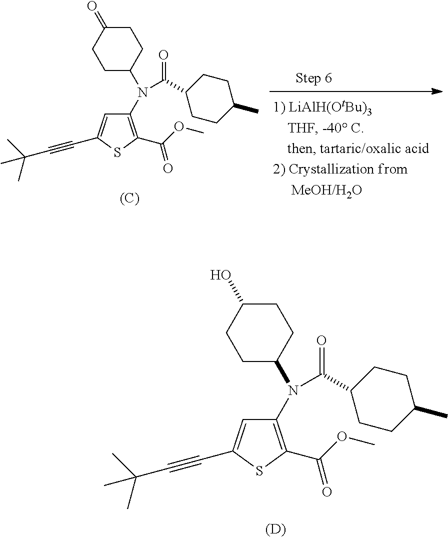

Compound (C) (399 g, 1.0 eq, limiting reagent) was charged to a 12 L reactor and purged with N₂. Anhydrous THF (2 L, 5.0 vol) was then charged to the reactor, then the mixture was agitated. The resulting solution was cooled to −65 to −64° C.

LiAlH(OtBu)₃ (960 ml of 1 M in THF, 2.40 vol or 1.1 eq) was added while maintaining not higher than −40° C. batch temperature. The solution was added over 2 hours and 15 minutes. The rate of addition was 1.45 vol/h.

Upon completion of LiAlH(OtBu)₃ addition, the batch was stirred at −40° C. or lower temperature for 1 additional hour. A small IPC sample was collected after 1 h and immediately quenched with 1 N HCl. The sample was analyzed for Compound (C) consumption (the reaction was judged complete when Compound (C) was ≦0.5% with respect to Compound (D) by IPC method).

If reaction was not completed, stir reaction at −40° C. for an additional hour. An IPC sample was collected and immediately quenched with 1 N HCl. If reaction was not completed, then additional amount of LiAlH(OtBu)₃ was added (for instance, if 1.0% peak area of unreacted Compound (C) remained compared to product Compound (D), then 2% of the original charge of LiAlH(OtBu)₃ solution was added). The batch was kept at −40 to −50° C. or lower temperature during reaction. Upon addition of LiAlH(OtBu)₃, the batch was stirred for 1 hour at −45 to −40° C. A small IPC sample was collected and immediately quenched with 1 N HCl.

Once the reaction was complete, MTBE (1197 L, 3 vol) was charged to the batch, then the batch was warmed to 0° C. The resulting solution was added over about 10-15 minutes to a mixture of aqueous oxalic acid (or tartaric acid) which was prepared by cooling a mixture of oxalic acid (or tartaric acid) (9% w/w, 2394 L, 6 vol) and MTBE (7 L, 2 vol) to 8-10° C. The batch temperature was adjusted to 15-25° C. and the resulting mixture was stirred for 30-60 minutes.

The agitation was stopped. The upper organic phase was collected. Water (2.8 L, 7 vol) was added to the organic phase. The biphasic mixture was stirred for 10 minutes at 15-25° C. Then agitation was stopped. The upper organic phase was collected.

Crystallization of Compound (D) was performed by switching solvent to methanol. The batch volume was reduced to 1.2 L or 3.0 vol by vacuum distillation at <60° C.

Methanol (4 L, 10 vol) was added to the batch (without adjusting batch temperature) and the batch volume was reduced to 1.2 L or 3.0 vol by vacuum distillation at <60° C. This step was repeated. Then, the batch volume was adjusted to 3.0 vol by addition of 479 mL.

A small IPC sample of the slurry was collected. The solids were filtered and the solution was analyzed by gas chromatography to determine the level of residual THF and MTBE with respect to methanol. If solvent switch to methanol was complete, then the batch was heated to 60-65° C. and stirred at this temperature until all solids dissolved. 2 volumes of the 50 vol % methanol/water solution was added, maintaining the temperature at not less than (NLT) 50° C. Then, the temperature was adjusted to 47-53° C. (e.g., 50° C.), and the temperature was maintained for 4 hours in order for solids to start crystallizing. Then, the remaining 2 volumes of the 50 vol % methanol/water solution were added into the batch. The batch was then cooled 15-25° C. at approximately 5° C./hour, and was held for not less than (NLT) 4 hours at 15-25° C. The filter cake was washed with 1 volume (based on compound 5 charge) of 50 volume % methanol/water

The material was dried for at least 12 hours under vacuum with nitrogen bleed at 55-65° C.

If required, the batch could be recrystallized by charging dry Compound (D) (1 equiv) and methanol (2 vol, relative to Compound (D) charge) to a reactor and heating the batch to 60-65° C. until all solids dissolved. The batch would then be cooled to −20° C. over a 3 hour period. The resulting solids would be filtered and dried for at least 12 hours under vacuum with nitrogen bleed at 55-65° C. Compound D: ¹H NMR (400 MHz, DMSO-d₆) δ 0.52-0.69 (m, 2H), 0.75 (d, 6.4 Hz, 3H), 0.76-0.86 (m, 1H), 1.11-1.24 (m, 5H), 1.31 (s, 9H), 1.43-1.57 (m, 6H), 1.73-1.83 (m, 4H), 3.17-3.18 (m, 1H), 3.75 (s, 3H), 4.24-4.30 (m, 1H), 4.49 (d, J=4.4 Hz, 1H), 7.23 (s, 1H).

B. Method B: Reducing Reagents Other than LiAlH(OtBu)₃

Reducing reagents other than LiAlH(OtBu)₃ that gave predominantly the desired isomer were: LiAlH(OiBu)₂(OtBu)₃, DiBAlH, LiBH4, NaBH4, NaBH(OAc)₃, Bu₄NBH₄, ADH005 MeOH/KRED recycle mix A, KRED-130 MeOH/KRED recycle mix A, Al(Oi-Pr)₃/i-PrOH, and (i-Bu)₂AlOiPr.

### 7. Step 7

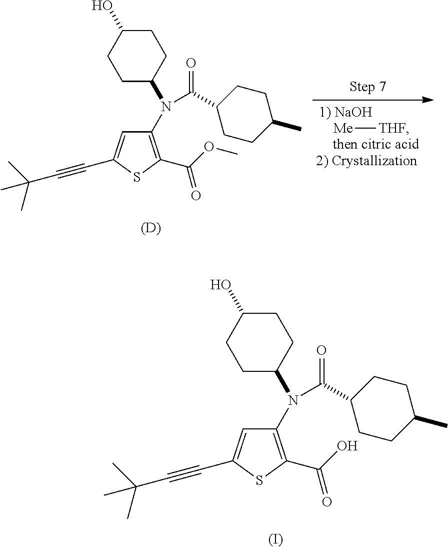

Compound (D) and Me-THF (5 volumes, based on compound 6 charge) were added to a reactor. To the solution, an aqueous solution of NaOH (2N, 4.0 vol, 3.7 equiv) was added at 15-25° C. The batch was heated to 68-72° C. and stirred for 8-16 hours at this temperature. The reaction progress was monitored by LC. Upon completion, the batch was cooled to 0-5° C. Precipitates formed. An aqueous solution of citric acid (30% by weight, 3.7 equiv), was added over 15-30 minutes, while maintaining the batch temperature below 25° C. The phases were separated. Water was added (5 volumes based on compound 6 charge) to the organic layer. The phases were separated. The batch volume was reduced to 3 volumes (based on compound (D) charge) via vacuum distillation at a maximum temperature of 35° C. Then dry Me-THF (3 vol, based on compound (D) charge) was added. The water content was determined by Karl Fisher titration. The batch is deemed dry if residual water level is ≦1.0%.

Optionally, the final product of Compound (1) can be recrystallized either in EtOAc or in a mixture of nBuOAc and acetone via solvent switch described below to form Form M of Compound (1):

A: Recrystallization in a Mixture of nBuOAc and Acetone:

A solvent switch from 2-Me-THF to nBuOAc was performed by first reducing the batch volume to 2-3 volumes (based on compound (D) charge) by vacuum distillation at a maximum temperature of 45° C. nBuOAc (3 vol, based on compound (D) charge) was added and the batch volume was reduced to 2-3 volumes (based on compound (D) charge) via vacuum distillation at a maximum temperature of 45° C. The batch volume was then adjusted to a total of 5-6 volumes by addition of nBuOAc. The solution was analyzed for residual 2-Me-THF in content in nBuOAc. This cycle was repeated until less than 1% of 2-Me-THF with respect to nBuOAc remained, as determined by GC analysis. Once the residual 2-Me-THF IPC criterion was met and it was insured that the total batch volume is 6 (based on compound (D) charge), the batch temperature was adjusted to 40-45° C. Acetone is then charged into the batch to have approximately 10 wt % acetone in the solvent. The batch temperature was adjusted to 40-45° C. Compound 1 seed (1.0% by weight with respect to the total target weight of compound (1)) was added. The batch was agitated at 40-45° C. for 4-8 hours. The recrystallization progress is monitored by X-ray powder diffraction (XRPD). If spectrogram matched that of required form, then the batch was cooled from 40-45° C. to 30-35° C. (preferably about 35° C.) at rate of 5° C./hour. The batch was held at about 35° C. for at least one hour, and then filtered and the filter cake was washed with 9:1 wt:wt mixture of nBuOAc/acetone (1 vol). The material was dried in vacuum with nitrogen bleed at NMT 45° C. for 12-24 hours. The expected isolated molar yield of compound (1) (Form M) starting with compound (D) was 80-85%. Compound (1): ¹H NMR (400 MHz, DMSO-d₆) δ0.58 (m, 1H), 0.74 (q, J=6.53 Hz, 1H), 0.81 (ddd, J=12.86, 12.49, 3.19 Hz, 1H), 1.18 (m, 5H), 1.28 (s, 3H), 1.42 (m, 1H), 1.55 (m, 3H), 1.61 (m, 1H), 1.73 (m, 2H), 1.81 (m, 2H), 3.19 (m, 1H), 4.26 (m, 1H), 4.49 (bs, 1H), 7.14 (s, 1H), 13.45 (bs, 1H).

### B: Recrystallization in EtOAc:

A solvent switch from 2-Me-THF to EtOAc was performed by first reducing the batch volume to 2-3 volumes (based on compound (D) charge) by vacuum distillation at a maximum temperature of 35° C. EtOAc (10 vol, based on compound (D) charge) was added and the batch volume was reduced to 2-3 volumes (based on compound (D) charge) via vacuum distillation at a maximum temperature of 35° C. The solution was analyzed for residual 2-Me-THF in content in EtOAc. This cycle was repeated until less than 1% of Me-THF with respect to EtOAc remained, as determined by GC analysis. Once the residual 2-Me-THF IPC criterion was met and it was insured that the total batch volume is 10 (based on compound (D) charge), the batch temperature was adjusted to 40-45° C. Compound 1 seed (1.0% by weight with respect to the total target weight of compound (1)) was added. The batch was agitated at 40-45° C. for 12 hours. A flat floor/flat bottomed reactor (not conical) should be used. The recrystallization progress is monitored by X-ray powder diffraction (XRPD). If spectrogram matched that of required form, then the batch was cooled from 40-45° C. to 11-14° C. at rate of 5° C./hour. The batch was filtered and the filter cake was washed with EtOAc (1 vol), previously chilled to 11-14° C. The material was dried in vacuum with nitrogen bleed at NMT 45° C. for 12-24 hours. The expected isolated molar yield of compound (1) (Form M) starting with compound (D) was 80-85%.

### Example 3

### Formation of Co-Crystals of Compound (1)

### 3A: Formation of Urea Co-Crystal

Method A

Urea co-crystals of Compound (1) can be prepared by following the steps described below:

10 mg of Compound (1) was charged to a reactor. 1.35 mg of urea (1:1 molar ratio) was then charged to the reactor. Into the reactor was added dichloromethane (0.5 mL). The reaction mixture was stirred at room temperature for 8 days to form urea co-crystals of Compound (1). The resulting solids of urea co-crystals of Compound (1) were filtered and dried.

Method B

Alternatively, urea co-crystals of Compound (1) can be prepared by following the steps described below:

75 mg of Compound (1) was charged to a reactor. 10.13 mg of urea (1:1 molar ratio) was then charged to the reactor. Into the reactor was added acetonitrile (20 mL). The reaction mixture was stirred at room temperature for a day to form urea co-crystals of Compound (1). The resulting solids of urea co-crystals of Compound (1) were filtered and dried.

Characteristics of urea co-crystals Compound (1): XRPD data of urea co-crystals of Compound (1) is shown in FIG. 1. Certain representative XRPD peaks and DSC endotherm (° C.) of urea co-crystals of Compound (1) are summarized in Table 1 below.

TABLE 1
Certain representative XRPD peaks and
DSC endotherm (° C.) of urea co-crystals of Compound (1)
Urea Cocrystal
DSC Endotherm (° C.) 190° C.

| XRPD Peaks | Angle (2-Theta ± 0.2) | Intensity % |
|---|---|---|
| 1 | 18.4 | 100.0 |
| 2 | 12.1 | 69.1 |
| 3 | 15.6 | 65.0 |
| 4 | 20.1 | 52.6 |
| 5 | 10.8 | 46.5 |
| 6 | 11.7 | 44.1 |

3B: Formation of Nicotinamide Co-crystal

Nicotinamide co-crystals of Compound (1) can be prepared by following the steps described below:

75 mg of Compound (1) was charged to a reactor. 16.13 mg of nicotinamide (1:1 molar ratio) was then charged to the reactor. Into the reactor was added acetonitrile (20 mL). The reaction mixture was stirred at room temperature for a day to form urea co-crystals of Compound (1). The resulting solids of urea co-crystals of Compound (1) were filtered and dried.

Characteristics of nicotinamide co-crystals Compound (1): XRPD data of nicotinamide co-crystals of Compound (1) is shown in FIG. 2. Certain representative XRPD peaks of nicotinamide co-crystals of Compound (1) are summarized in Table 2 below.

TABLE 2
Certain representative XRPD peaks) of nicotinamide co-crystals of
Compound (1)
Nicotinamide Cocrystal

| XRPD Peaks | Angle (2-Theta ± 0.2) | Intensity % |
|---|---|---|
| 1 | 21.7 | 100.0 |
| 2 | 10.2 | 54.8 |
| 3 | 18.9 | 53.2 |
| 4 | 17.8 | 50.4 |
| 5 | 22.9 | 44.6 |
| 6 | 15.5 | 42.5 |

3C: Formation of Nicotinamide Co-Crystal

Isonicotinamide co-crystals of Compound (1) can be prepared by following the steps described below:

75 mg of Compound (1) was charged to a reactor. 16.13 mg of isonicotinamide (1:1 molar ratio) was then charged to the reactor. Into the reactor was added acetonitrile (20 mL). The reaction mixture was stirred at room temperature for a day to form urea co-crystals of Compound (1). The resulting solids of urea co-crystals of Compound (1) were filtered and dried.

Characteristics of isonicotinamide co-crystals Compound (1): XRPD data of isonicotinamide co-crystals of Compound (1) is shown in FIG. 3. Certain representative XRPD peaks of isonicotinamide co-crystals of Compound (1) are summarized in Table 3 below.

TABLE 3
Certain representative XRPD peaks of isonicotinamide co-crystals of
Compound (1)
Isonicotinamide
Cocrystal

| XRPD Peaks | Angle (2-Theta ± 0.2) | Intensity % |
|---|---|---|
| 1 | 21.7 | 100.0 |
| 2 | 10.2 | 63.6 |
| 3 | 17.8 | 32.8 |
| 4 | 22.9 | 28.9 |
| 5 | 18.9 | 27.8 |
| 6 | 11.6 | 23.8 |

All references provided herein are incorporated herein in its entirety by reference. As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A co-crystal comprising Compound (1) and a co-crystal former selected from the group consisting of urea, nicotinamide, and isonicotinamide, wherein Compound (1) is represented by the following structural formula:

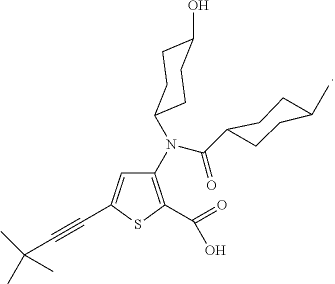 [SMILES: CC1CCC(C(=O)N(c2cc(C#CC(C)(C)C)sc2C(=O)O)C2CCC(O)CC2)CC1]

2. The co-crystal of claim 1, wherein the co-crystal former is urea.

3. The co-crystal of claim 2, characterized as having an X-ray powder diffraction pattern with characteristic peaks expressed in 2-theta±0.2 at the following positions: 18.4, 12.1, 15.6, 20.1, 10.8, and 11.7, wherein the X-ray powder diffraction pattern is obtained at room temperature using Cu K alpha radiation.

4. The co-crystal of claim 2, wherein the X-ray powder diffraction pattern includes characteristic peaks expressed in 2-theta±0.2 at the following positions with relative intensities in parentheses: 18.4 (100.0%), 12.1 (69.1%), 15.6 (65.0%), 20.1 (52.6%), 10.8 (46.5%), and 11.7 (44.1%).

5. The co-crystal of claim 2, characterized as having an endothermic peak in differential scanning calorimetry (DSC) at 190±2° C.

6. The co-crystal of claim 2, characterized as having X-ray powder diffraction pattern substantially the same as that shown in FIG. 1.

7. The co-crystal of claim 1, wherein the co-crystal former is nicotinamide.

8. The co-crystal of claim 7, characterized as having an X-ray powder diffraction pattern with characteristic peaks expressed in 2-theta±0.2 at the following positions: 21.7 and 15.5, wherein the X-ray powder diffraction pattern is obtained at room temperature using Cu K alpha radiation.

9. The co-crystal of claim 8, characterized as having an X-ray powder diffraction pattern with characteristic peaks expressed in 2-theta±0.2 at the following positions: 21.7, 10.2, 18.9, 17.8, 22.9, and 15.5, wherein the X-ray powder diffraction pattern is obtained at room temperature using Cu K alpha radiation.

10. The co-crystal of claim 9, wherein the X-ray powder diffraction pattern includes characteristic peaks expressed in 2-theta±0.2 at the following positions with relative intensities in parentheses: 21.7 (100.0%), 10.2 (54.8%), 18.9 (53.2%), 17.8 (50.4%), 22.9 (44.6%), and 15.5 (42.5%).

11. The co-crystal of claim 7, characterized as having X-ray powder diffraction pattern substantially the same as that shown in FIG. 2.

12. The co-crystal of claim 1, wherein the co-crystal former is isonicotinamide.

13. The co-crystal of claim 12, characterized as having an X-ray powder diffraction pattern with characteristic peaks expressed in 2-theta±0.2 at the following positions: 21.7 and 11.6, wherein the X-ray powder diffraction pattern is obtained at room temperature using Cu K alpha radiation.

14. The co-crystal of claim 13, characterized as having an X-ray powder diffraction pattern with characteristic peaks expressed in 2-theta±0.2 at the following positions: 21.7, 10.2, 17.8, 22.9, 18.9, and 11.6, wherein the X-ray powder diffraction pattern is obtained at room temperature using Cu K alpha radiation.

15. The co-crystal of claim 14, wherein the X-ray powder diffraction pattern includes characteristic peaks expressed in 2-theta±0.2 at the following positions with relative intensities in parentheses: 21.7 (100.0%), 10.2 (63.6%), 17.8 (32.8%), 22.9 (28.9%), 18.9 (27.8%), and 11.6 (23.8%).

16. The co-crystal of claim 12, characterized as having X-ray powder diffraction pattern substantially the same as that shown in FIG. 3.

17. A pharmaceutical composition comprising a co-crystal of any one of claims 1-16 and at least one pharmaceutically acceptable carrier or excipient.

18. A method of inhibiting or reducing the activity of HCV polymerase in a biological in vitro sample, comprising administering to the sample an effective amount of a co-crystal according to any one of claims 1-16.

19. A method of treating a HCV infection in a subject, comprising administering to the subject a therapeutically effective amount of a co-crystal according to any one of claims 1-16.

20. A method of inhibiting or reducing the activity of HCV polymerase in a subject, comprising administering to the subject a therapeutically effective amount of a co-crystal according to any one of claims 1-16.

21. The method of claim 19 or 20, further comprising co-administering one or more additional therapeutic agents to the subject.

22. The method of claim 21, wherein the additional therapeutic agents include an anti-HCV drug.

23. The method of claim 22, wherein the anti-HCV drug is an HCV protease inhibitor.

24. The method of claim 23, wherein the HCV protease inhibitor is an HCV NS3 inhibitor.

25. The method of claim 21, wherein the anti-HCV drug is an HCV NS5A inhibitor.

26. The method of any one of claims 21-25, wherein an interferon and/or ribavirin is co-administered.

27. The method of claim 26, wherein the interferon is a pegylated interferon.

28. The method of claim 27, wherein the pegylated interferon is a pegylated interferon-alpha.

29. The method of claim 27, wherein the pegylated interferon is pegylated interferon-alpha 2a or pegylated interferon-alpha 2b.

30. The method of any one of claims 18-29, wherein the HCV is genotype 1.

31. The method of any one of claims 18-29, wherein the HCV is genotype 1a or genotype 1b.

32. A method of preparing a co-crystal comprising Compound (1) and a co-crystal former selected from the group consisting of urea, nicotinamide, isonicotinamide, wherein Compound (1) is represented by the following structural formula: 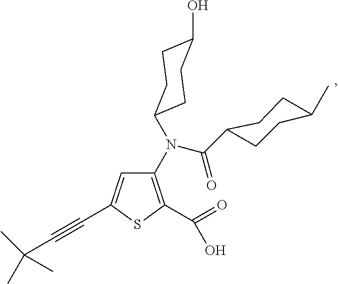 [SMILES: CC1CCC(C(=O)N(c2cc(C#CC(C)(C)C)sc2C(=O)O)C2CCC(O)CC2)CC1]
comprising the step of:
stirring a mixture of Compound 1 and the co-crystal former to form the co-crystal.

33. The method of claim 32, wherein the co-crystal former is urea.

34. The method of claim 33, wherein the mixture of Compound 1 and the co-crystal former is stirred in a solvent system that includes dichloromethane and/or acetonitrile.

35. The method of claim 32, wherein the co-crystal former is nicotinamide.

36. The method of claim 35, wherein the solvent system includes acetonitrile.

37. The method of claim 32, wherein the co-crystal former is isonicotinamide.

38. The method of claim 37, wherein the solvent system includes acetonitrile.
